SEQUENCE LISTING (20 sequences)

SEQ ID NO: 1 - RNA, 22 nt, Homo sapiens
acaggugagguucuugggagcc

SEQ ID NO: 2 - RNA, 22 nt, Homo sapiens
cagugcaaugaugaaagggcau

SEQ ID NO: 3 - RNA, 22 nt, Homo sapiens
aaguucuguuauacacucaggc

SEQ ID NO: 4 - RNA, 21 nt, Homo sapiens
uagcagcacagaaauauuggc

SEQ ID NO: 5 - RNA, 22 nt, Homo sapiens
auagcagcaugaaccugucuca

SEQ ID NO: 6 - RNA, 22 nt, Homo sapiens
aaugcacccgggcaaggauucu

SEQ ID NO: 7 - RNA, 22 nt, Homo sapiens
aacccguagauccgaacuugug

SEQ ID NO: 8 - RNA, 23 nt, Homo sapiens
agguuacccgagcaacuuugcau

SEQ ID NO: 9 - RNA, 22 nt, Homo sapiens
cuuggcaccuagcaagcacuca

SEQ ID NO: 10 - RNA, 24 nt, Homo sapiens
uugcagcugccugggagugacuuc

SEQ ID NO: 11 - RNA, 22 nt, Homo sapiens
cacccguagaaccgaccuugcg

SEQ ID NO: 12 - RNA, 22 nt, Homo sapiens
ucggauccgucugagcuuggcu

SEQ ID NO: 13 - RNA, 22 nt, Homo sapiens
uggugggccgcagaacaugugc

SEQ ID NO: 14 - RNA, 22 nt, Homo sapiens
ugcggggcuagggcuaacagca

SEQ ID NO: 15 - RNA, 22 nt, Homo sapiens
uggguggucuggagauuugugc

SEQ ID NO: 16 - RNA, 21 nt, Homo sapiens
aauauaacacagauggccugu

SEQ ID NO: 17 - RNA, 22 nt, Homo sapiens
aaugcaccugggcaaggauuca

SEQ ID NO: 18 - RNA, 22 nt, Homo sapiens
ugaaacauacacgggaaaccuc

SEQ ID NO: 19 - DNA, 20 bp, Artificial Sequence
  synthetic miR-130b-3p target sequence
tcacgttgtccctttctcac

SEQ ID NO: 20 - DNA, 1419 bp, Homo sapiens
aactggactaaaccttttagacactgtaccaggatacagggtcacaggcctgttgatattgagatgtaaacccagaatattcttgcattacacagaaacaccattgcaacaccagctacactccacatcctacagaaaagcattacccattttgcggccaagttcccgacgagaggctggacctcttcatcacattgacttacgccgttgcttttccagactgggcagaggggctgacttcgcagtgtgtgccaaagagccggtgtctgataatcccattttcctgcttatcacctgaactgtgtcagtatcacttttagttttgttggttggttggtttgttgtttgtttaatatgccctgttttctacttctgttggaaaatatttggggttgaaataaaccagtgggagcatgggagccagtttggtggttggcaaactaccagtgatagaaaacaaatgacaggttttctgtgagcgtacgtcacagtggctcgggcccacaaggagacagaggggtacgtttcaggacatcattccaagggtttctgttcacgtttgtttaacgaggaaggggaagttgttctcccataataccagtggtatttctagtgcaacccgggtgtttttctacgcctcttcccatgctgcttccccacccccccactgtgctccgccccttccaaatgccatgtcacaacagcacttggatgtgtttttctcaactgtcatcagctccagctggcaggaccaacttcttgaaacacaggaagcatccagcggaaaatattttaataaaacagactcctcataaaatattgttcgggggagggggaagaaaccggctccaccaatctgtcggcattgtattgggagatgtgagaagccaggctggcagcaggggcccgatcctaatgggcctttgtcaggtggtggttccgtgttgggcacacggacttggtgcctggccctttaatctggcctgggttttcttgaatcacagatcttcatctccactgggaatatcaaagtgcttgggaaacttagaagactggggcagcagaaggggatgcaggatctttgaagtggagaggagagattcattcctcctgacttcttgccccaactctcacttccaattgcacattaaataatcccagcactttgggaggccaaggcgagcggatcacctgaggtcaggggttcgagaccagcctgggcaacatggcgaaaccccgtctctactaaaaatacaaaaattagccgagtgtggtggcgcacccctgtaattccagctactcgggaggctgaggcatgagaatcacttgaacccaggaggcggaggctgccgtgagctgagatcacaccaccacactccagcctgtgcaacagagtgagactctgtctcaaaaataaaa

(19) United States
(12) Patent Application Publication
(10) Pub. No.: US 2022/0143067 A1
(43) Pub. Date: May 12, 2022

(54) METHODS FOR DIAGNOSIS AND/OR THERAPY OF DISORDERS THAT CORRELATE WITH DENNDIA VARIANT 2

(71) Applicants: Virginia Commonwealth University, Richmond, VA (US); The Penn State Research Foundation, University Park, PA (US)

(72) Inventors: Jerome F. STRAUSS, Richmond, VA (US); Janette M. MCALLISTER, Richmond, VA (US)

(21) Appl. No.: 17/431,887

(22) PCT Filed: Feb. 20, 2020
(86) PCT No.: PCT/US2020/018935

§ 371 Date: Aug. 18, 2021

Related U.S. Application Data
(60) Provisional application No. 62/808,086, filed on Feb. 20, 2019.

(51) Int. Cl. A61K 31/7105; C07K 16/18; A61P 15/08; C12Q 1/6883; C12Q 1/6851

(52) U.S. Cl. CPC A61K 31/7105; C07K 16/18; C12Q 2600/178; C12Q 1/6883; C12Q 1/6851; A61P 15/08

(57) ABSTRACT

Methods for the diagnosis and treatment of DENND1A. V2 related disorders, such as PCOS, are provided. In particular, microRNAs which are upregulated or downregulated in DENND1A. V2 related disorders and methods for using the same are provided.

### STATEMENT REGARDING FEDERALLY SPONSORED RESEARCH OR DEVELOPMENT

This invention was made with government support under grant number R01 HD083323 awarded by the National Institutes of Health (NIH). The government has certain rights in the invention.

### FIELD

The present disclosure relates generally to methods for diagnosing and treating disorders that correlate with expression of DENND1A. V2 (DENN/MADD domain containing 1A variant 2) mRNA and/or protein, including without limitation polycystic ovary syndrome (PCOS).

### BACKGROUND

Polycystic ovary syndrome (PCOS) is a common endocrine disorder that affects 5%-10% of reproductive age women across multiple ethnic groups world-wide¹⁻⁷, and is by far the most common disorder involving overproduction of sex steroids⁸. The disorder is heterogeneous, characterized by a broad spectrum of reproductive, metabolic and endocrinological features including hyperandrogenism, anovulation, infertility, and the presence of multiple small subcortical follicular cysts embedded in bilaterally enlarged ovaries^{9,10}. Other endocrinopathies and metabolic features commonly observed in PCOS patients include abnormal glucose metabolism, insulin resistance, dyslipidemia and obesity. PCOS women are known to have higher prevalence rates of type 2 diabetes (T2D), metabolic syndrome and cardiovascular disease^{1,11}. Family-based studies revealed that PCOS is a complex genetic disorder^{12,13} There is also evidence that strongly suggests that epigenetic and post-translational gene regulatory factors influence the complex pathophysiology of PCOS¹⁴⁻¹⁹ Most consensus diagnosis schemes recommend the presence of hyperandrogenism (clinical or biochemical) as an essential characteristic for reaching a PCOS diagnosis, and it is considered the hallmark of PCOS. Cross-sectional case-control studies suggested that hyperandrogenism is implicated in both the metabolic and reproductive morbidities of PCOS, and may be a common link between the two²⁰⁻²².

Ovarian androgen production is increased in PCOS, and studies have shown that theca cells from PCOS women secrete more androgen than theca cells from regularly ovulating women²³⁻²⁸. Thus, understanding gene expression and its regulation in theca cells is central to understanding PCOS pathophysiology, or at least hyperandrogenism associated with PCOS. Wood et al.^{29, 30} demonstrated that PCOS theca cells have a characteristic molecular signature as compared to normal theca cells. Although these results revealed altered gene expression in PCOS theca as compared to normal theca cells, it is not yet known whether the altered expression profile is the result of transcriptional or post-transcriptional regulatory mechanisms.

MicroRNAs (miRNAs) are small (20-24 nucleotides) single-stranded, noncoding, regulatory RNA molecules. They are involved in post-transcriptional regulation of gene expression either by complementary binding to the 3′-untranslated region of their target mRNA, thereby inhibiting translation³¹, or by inducing mRNA degradation³². The post-transcriptional mechanism operationalized for down regulation of gene expression depends upon the binding target sequence. Messenger RNA degradation occurs in the case of complete or near complete complementarity between miRNA and the target, while repression or inhibition of translation occurs if there is not sufficient complementarity for cleavage³². Although not much is known about the roles of miRNA in the hyperandrogenemia of PCOS, there have been studies providing evidence of differential miRNA expression in the ovarian stroma, endometrium, follicular fluid, serum, and granulosa cells of women with PCOS^{33-45,92-96}. Metformin, an insulin-sensitizing drug, which is used as a treatment to lower insulin and androgen levels and initiate ovulation in women with PCOS, changes global miRNA expression patterns⁴⁶⁻⁴⁸. MicroRNAs are also reported to be stably expressed in encapsulated vesicles or free-circulating in the serum, plasma, urine and saliva^{49, 50}. Although a recent study compared the differential expression of a cohort of miRNAs using a limited miRNA-microarray platform in intact theca cells isolated from wedge resections of ovaries of normal cycling and PCOS women⁵¹, there are no reports that have explored differential miRNA expression in normal and PCOS theca cells using global miRNA deep-sequencing combined with functional analyses, to identify miRNA that underlie increased androgen biosynthesis and gene expression in PCOS.

Several genome-wide association studies (GWAS) have identified loci significantly associated with PCOS. In a two-part study, GWAS on Han-Chinese populations identified 11 candidate loci^{52,53}. Subsequent GWAS on European populations confirmed associations of some of the Han-Chinese GWAS to PCOS, and also identified additional candidate loci^{54, 55}. Together, four GWAS^{52,53,56-59} and PCOS GWAS meta-analyses⁶⁰, have identified 22 candidate loci/genes for PCOS, including DENND1A. Although some of these loci consist of plausible PCOS candidate genes, the differential expression of the specific isoforms of these genes has not been examined, and the molecular mechanisms by which most of the identified genes contribute to PCOS are not clearly understood. It was reported that a truncated splice variant of DENND1A, DENND1A. V2, is increased in PCOS theca cells, with no change in DENND1A. V1 (full-length isoform) expression⁶¹. Forced expression of DENND1A. V2 in normal theca cells increased 17α-hydroxylase (CYP17A1) expression and androgen production, whereas knockdown of DENND1A. V2 expression in PCOS theca cells reduced CYP17A1 expression androgen synthesis, implicating DENND1A. V2 in the regulation of steroidogenesis and the PCOS phenotype⁶¹. The mechanisms underlying the increased DENND1A. V2 expression in PCOS theca cells, as well as their altered transcriptome signature in PCOS, remain to be elucidated. Moreover, the role of differentially expressed microRNAs in PCOS theca cells on increased DENND1A. V2 and CYP17A1 mRNA and augmented androgen biosynthesis has not been examined.

### SUMMARY

In the present disclosure, data are presented in which the microRNA expression profiles of human theca cell cultures established from women with PCOS and without the disease were determined using next generation small RNA deep-sequencing. Target gene analysis of the differentially expressed microRNA was focused on PCOS candidate genes identified by GWAS⁵²⁻⁵⁵. These studies provide the first evidence that differential expression of microRNAs such as miR-130b-3p in normal and PCOS theca cells and serum is associated with the expression of DENND1A. V2 and CYP17A1 mRNA and androgen biosynthesis. IPA Core Pathway and Network Analyses identified a common network whereby miR-130b-3p's predicted interactions with the PCOS GWAS candidates DENND1A. V2, LHCGR, and RAB5B, as well as signaling components that mediate the increased gene expression required for hyperandrogenemia.

One aspect of the disclosure provides a method for diagnosis of a disorder that is positively correlated with expression of DENN/MADD domain containing 1A variant 2 (DENND1A. V2) mRNA and/or protein in a subject comprising measuring an expression level of at least one miRNA selected from the group consisting of miR-501-3p, miR-100-5p, miR-409-5p, miR-125a-3p, miR-1271-5p, miR-1301-3p, miR-130b-3p, miR-99b-5p, miR-127-3p, miR-148b-5p, miR-654-5p, miR195-5p, miR-744-5p, miR-1293, miR-410-3p, miR-4524a-5p, miR-502-3p, and miR-494-3p in a biological sample from said subject; and comparing the expression level of the at least one miRNA to a corresponding reference value. In some embodiments, the at least one miRNA includes miR-130b-3p.

In some embodiments, the corresponding reference value is obtained from one or more healthy subjects. In some embodiments, the method further comprises determining that the subject has the disorder when the expression level of one or more of miR-125a-3p, miR-130b-3p, miR-148b-5p, miR195-5p, and miR-4524a-5p is lower than the corresponding reference value; and/or determining that the subject has the disorder when the expression level of one or more of miR-501-3p, miR-100-5p, miR-409-5p, miR-1271-5p, miR-1301-3p, miR-99b-5p, miR-127-3p, miR-654-5p, miR-744-5p, miR-1293, miR-410-3p, miR-502-3p, and miR-494-3p is higher than the corresponding reference value when the biological sample is not serum and vice versa when the biological sample is serum. In some embodiments, the disorder is polycystic ovary syndrome (PCOS). In some embodiments, the biological sample is blood, serum, urine, plasma, or saliva and/or a biopsy or surgical specimen of the ovary, endometrium, adipose tissue, or skeletal muscle.

Another aspect of the disclosure provides a method for diagnosis of a disorder that is positively correlated with expression of DENND1A. V2 mRNA and/or protein in a subject comprising measuring an expression level of DENND1A. V2 mRNA and miR-130b-3p in a biological sample from said subject; determining a ratio of the expression level of DENND1A. V2 mRNA to the expression level of miR-130b-3p; and comparing the ratio to a corresponding reference ratio. In some embodiments, the corresponding reference ratio is obtained from one or more healthy subjects. In some embodiments, the method further comprises determining that the subject has the disorder when the ratio is higher than the corresponding reference ratio when the biological sample is not serum and vice versa when the biological sample is serum. In some embodiments, the disorder is PCOS. In some embodiments, the biological sample is blood, serum, urine, plasma, or saliva and/or a biopsy or surgical specimen of the ovary, endometrium, adipose tissue, or skeletal muscle, and may be dependent on whether a specific compartmental component (i.e, endosomal/exosomal) is examined.

Another aspect of the disclosure provides a method for the treatment of a disorder that is positively correlated with expression of DENND1A. V2 mRNA and/or protein comprising measuring an expression level of at least one miRNA selected from the group consisting of miR-501-3p, miR-100-5p, miR-409-5p, miR-125a-3p, miR-1271-5p, miR-1301-3p, miR-130b-3p, miR-99b-5p, miR-127-3p, miR-148b-5p, miR-654-5p, miR195-5p, miR-744-5p, miR-1293, miR-410-3p, miR-4524a-5p, miR-502-3p, and miR-494-3p in a biological sample from said subject; comparing the expression level of the at least one miRNA to a corresponding reference value; determining that the subject has the disorder when the expression level of one or more of miR-125a-3p, miR-130b-3p, miR-148b-5p, miR-195-5p, and miR-4524a-5p is lower than the corresponding reference value; and/or the expression level of one or more of miR-501-3p, miR-100-5p, miR-409-5p, miR-1271-5p, miR-1301-3p, miR-99b-5p, miR-127-3p, miR-654-5p, miR-744-5p, miR-1293, miR-410-3p, miR-502-3p, and miR-494-3p is higher than the corresponding reference value when the biological sample is not serum and vice versa when the biological sample is serum; and administering to the subject determined to have the disorder an appropriate treatment for the disorder. In some embodiments, the disorder is PCOS. In some embodiments, the treatment is selected from the group consisting of hormonal therapy, an insulin-sensitizing agent, an agent or surgical intervention to increase fertility, an antibody or an antigen binding fragment thereof that specifically recognizes DENND1A. V2 protein, and a RNAi containing composition, wherein said RNAi composition decreases the expression of DENND1A. V2. In some embodiments, the biological sample is blood, serum, urine, plasma, or saliva and/or a biopsy or surgical specimen of the ovary, endometrium, adipose tissue, or skeletal muscle.

Another aspect of the disclosure provides a method for the treatment of a disorder that is positively correlated with expression of DENND1A. V2 mRNA and/or protein comprising measuring an expression level of DENND1A. V2 mRNA and miR-130b-3p in a biological sample from said subject; determining a ratio of the expression level of DENND1A. V2 mRNA to the expression level of miR-130b-3p; comparing the ratio to a corresponding reference ratio; determining that the subject has the disorder when the ratio is higher than the corresponding reference ratio when the biological sample is not serum (which may include other biological fluids such as urine or plasma) and vice versa when the biological sample is serum; and administering to the subject determined to have the disorder an appropriate treatment for the disorder. In some embodiments, the disorder is PCOS. In some embodiments, the treatment is selected from the group consisting of hormonal therapy, an insulin-sensitizing agent, an agent or surgical intervention to increase fertility, an antibody or an antigen binding fragment thereof that specifically recognizes DENND1A. V2 protein, and a RNAi containing composition, wherein said RNAi (e.g., miRNA) composition decreases the expression of DENND1A. V2. In some embodiments, the biological sample is blood, serum, urine, plasma, or saliva and/or a biopsy or surgical specimen of the ovary, endometrium, adipose tissue, or skeletal muscle.

Other features and advantages of the present disclosure will be set forth in the description that follows, and in part will be apparent from the description or may be learned by practice of the disclosure.

### BRIEF DESCRIPTION OF THE DRAWINGS

FIG. 1. Heat-Map and hierarchical clustering of the most highly significant 18 differentially expressed microRNAs in normal and PCOS theca cells. Expression profiling of relative gene expression of the 18 microRNAs found to have the most significant differences between theca cells propagated from 4 normal cycling (NJ-4) and PCOS (P1-4) women (P<0.001) treated in the absence (C) and presence (F) of 20 μM forskolin. Variations in darker shades indicate increases in relative miR-RNA expression (>2.0), whereas variations in lighter shades indicate decreases (<2.0) in relative microRNA expression. Hierarchical clustering of microRNA expression shown by the bars to the left, suggests that 2 separate related families of microRNAs are involved in differential microRNA expression in normal and PCOS theca cells.

FIG. 2A-B. MicroRNA-130b-3p expression is decreased and DENND1A. V2 expression is increased in PCOS theca cells. A: To validate the decrease in expression of miR-130b-3p observed from microRNA expression profiling analysis, miR-130b-3p expression was analyzed by Real-Time qRT-PCR in theca cells propagated from 7 individual normal and 7 individual PCOS women treated in the absence (C) and presence (F) of 20 μM forskolin for 16 h. miR-130b-3p expression was decreased in PCOS theca cells as compared to normal theca cells under basal conditions (**P<0.01) and forskolin-stimulated conditions (*P<0.05). B: Utilizing mRNA harvested from the identical patients examined above, as we previously reported DENND1A. V2 expression was increased in PCOS theca cells, compared to normal theca cells under both normal (**P<0.001) and forskolin (***P<0.0001) stimulated conditions.

FIG. 3. MicroRNA target analyses provided evidence that mir-130b-3p targets the DENND1A. V2 3′UTR. A miR-130b-3p seed sequence (highlighted in middle of sequence) was identified approximately 527 bp past the DENND1A. V2 CDS stop codon (ATTGA), within its distinct 3′ untranslated region (UTR) using a combination of TargetScan, IPA Confidence, and Target-Miner analyses. The DENND1A. V2 polyadenylation signal AAAATAAAA is highlighted at the end of the sequence, approximately 1221 bp 3′ of the DENND1A. V2 CDS stop codon.

FIG. 4A-D. Decreased miR-130b-3p expression is correlated with increased DENND1A. V2 mRNA expression, and augmented CYP17A1 mRNA expression and DHEA biosynthesis. Real-time qRT-PCR analyses of miR-130b-3p for each of the individual normal (n=7) and PCOS (n=7) theca cell preparations, was compared with DENND1A. V2 (FIG. 4A), DENND1A. V1 (FIG. 4B), CYP17A1 (FIG. 4C) mRNA expression, and DHEA biosynthesis (FIG. 4D). A: There was a significant negative correlation between miR-130b-3p and DENND1A. V2 expression (Spearman's ρ=−0.6796, P<0.001). B: There was a significant positive relationship between miR-130b-3p and DENND1A. V1 mRNA expression (Spearman's ρ=0.3853, P=0.0429). C: There was significant negative correlation between miR-130b-3p and CYP17A1 mRNA abundance (Spearman's ρ=−0.6034, P=0.0001), and D: A significant negative correlation between miR-130b-3p levels and DHEA accumulation was observed in PCOS theca cells. (Spearman's ρ=−0.6836, P<0.0001).

FIG. 5A-C. MicroRNA-130b-3p overexpression in H295R cells downregulates DENND1A. V2 and CYP17A1 mRNA expression but has no effect on DENND1A. V1 mRNA expression. To evaluate the effects of miR-130b-3p mimic on DENND1A. V2, DENND1A. V1, and CYP17A1 mRNA expression, highly androgenic Adrenocortical H295R cells were transfected with 75 pM of either miR-130b-3p mimic or negative control mimic and treated with and without 20 μM forskolin for 48 h. A: Real-time qRT-PCR analyses of total mRNA harvested following transfection demonstrated that miR-130b-3p overexpression results in the down-regulation of DENND1A. V2 mRNA expression in control (*, P<0.05) and forskolin treated cells (***, P<0.001) as compared to Neg-mimic control. B: No significant differences in DENND1A. V1 mRNA expression were observed between Neg-mimic or miRNA-mimic transfected cells. C: CYP17A1 mRNA expression was decreased in miR-130b-3p transfected cells compared to Neg mimic control in forskolin treated cells (****, P<0.0005). Forskolin treatment also increased CYP17A1 mRNA in Neg-mimic cells (δ, P<0.0001).

FIG. 6A-C. Differences in the time courses of DENND1A. V2 and CYP17A1 mRNA accumulation in Adrenocortical H295R cells transfected with miR-130b-3p mimic. To explore the possibility that miR-130 mimic has temporal effects on DENND1A. V2, DENND1A. V1, and CYP17A1 mRNA expression in H295 cells, H295R cells were transfected with 75 pM of miR-130b-3p mimic or non-specific Neg-mimic, then subsequently treated in the absence (C) and presence of 20 μM forskolin (F). Following 24 and 48 hrs of treatments, mRNA was harvested and DENND1A. V2, DENND1A. V1, and CYP17A1 mRNA accumulation was assessed by qRT-PCR. The results are cumulative data from 4 replicative experiments in H295R cells. A: At both 24 and 48 hours following treatment, miR-130b-3p mimic significantly suppresses DENND1A. V2 mRNA under both control non-stimulated (24 h: ****, P<0.0001; 48 h: *, P<0.01) and forskolin-stimulated (24 h: *, P<0.01: 48 h: ***, P<0.001) conditions. B: The time course of DENND1A. V1 accumulation was unaffected by miR-130-3p mimic transfection. C: Of significant interest, CYP17A1 mRNA accumulation was increased by forskolin at both 24 h and 48 h (6, P<0.001) in Neg mimic transfected cells. miR-130 mimic transfection had no significant effect on CYP17A1 mRNA following 24 hrs of treatment; however, following 48 hrs of treatment, CYP17A1 mRNA was significantly suppressed (****, P<0.0001). These data demonstrate that miR-130b-3p mimic suppresses DENND1A. V2 mRNA at an earlier time point (24 h) than CYP17A1 mRNA (48 h).

FIG. 7A-D. Western analysis of DENND1A. V2 and DENND1A. V1 protein expression following hsa-miR-130b-3p mimic transfection of the human adrenocortical H295R cell line. Panel A: Representative Western analysis of ˜62 kD DENND1A. V2 and ˜119 kD DENND1A. V1 in whole cell extracts isolated from H295R cells transfected with 75 pM of miR-130b-3p mimic, or non-specific Neg mimic, treated in the absence (C) and presence (F) of 20 μM forskolin for 48 h. mTOR was used for total protein normalization. This data is representative of data obtained from 4 individual replicate experiments. Quantitative Western Blot data from 4 replicate experiments of H295R cells transfected with 75 pM miR-130b-3p and Neg mimics for 48 h, presented as the mean+/−SEM of the relative protein abundance normalized by mTOR of DENND1A. V2 (B) and DENND1A. V1 (C). Panel B: DENND1A. V2 protein was reduced following transfection with miR-130b-3p as compared to Neg-mimic under control (**, P<0.001) and forskolin-stimulated (**, P<0.005) conditions. Panel C: DENND1A. V1 protein was not significantly different following miR-mimic transfection in non-stimulated cells but increased by miR-130b-3p in forskolin treated cells (**, P<0.001). Panel D: The ratio of DENND1A. V2/V1 was reduced following transfection with miR-130b-3p mimic under control (***, P<0.001) and forskolin-stimulated (**, P<0.01) conditions, as compared to Neg-mimic. Both the relative abundance of DENND1A. V2 protein (B) and the ratio of DENND1A. V2/V1 protein (D) decreased in forskolin-stimulated H295R cells (δ, P<0.001) transfected with control Neg-mimic. In contrast, forskolin-treatment significantly increased DENND1A. V1 protein under both Neg-mimic (δ, P<0.005) and miR-130b-3p mimic (0, P<0.0001) conditions.

FIG. 8. IPA Network. Ingenuity pathway analyses suggest a common network between miR-130b-3p and PCOS GWAS candidate genes DENND1A, LHCGR, RAB5B and various signaling pathways in normal and PCOS theca cells that regulate androgen biosynthesis. We present a diagram of the resulting IPA network between miR-130b-3p, DENND1A; and the two other PCOS GWAS candidates (i.e., LHCGR, RAB5B), and the signaling component MAPK1 which were predicted to be targets of miR-130b-3p. IPA core pathway and network analyses also identified several signaling pathway components (Pka, PI3K) and transcription factors (GATA4/6) to be associated with augmented CYP17A1, CYP11A1, and HSB3B2 gene expression and excess androgen production in PCOS theca cells. The resulting theca cell cellular network provides an illustration of the possible mechanism(s) by which decreased miR-130b-3p in PCOS theca cells may mediate the PCOS GWAS candidates DENND1A, as well as LHCGR, and RAB5B, in addition to their associated signaling components and transcription factors resulting in increased CYP17A1, CYP11A1, and HSD3B2 gene expression and hyperandrogenism in PCOS theca cells.

FIG. 9. Comparison of the relative abundance of hsa-miR130b-3p in total RNA isolated from serum collected from nine PCOS and fifteen normally cycling women demonstrated a significant (****, P<0.0001) increase in expression in PCOS as compared to normal women.

### DETAILED DESCRIPTION

Embodiments of the disclosure relate to methods for diagnosis, prophylaxis, and/or therapy of a disorder that is positively correlated with expression of DENND1A. V2 mRNA and/or protein, such as PCOS. The methods include measuring the expression level of one or more microRNAs that were found to be differentially expressed in subjects having a disorder such as PCOS. microRNAs (miRNAs) can reduce mRNA levels by binding to specific target sequences in mRNAs leading to mRNA destruction or preventing of their translation. Thus, embodiments of the disclosure provide a non-invasive diagnostic test based on the quantitation of microRNAs in a biological sample such as urine. In some embodiments, the expression level of DENND1A. V2 mRNA, or another gene associated with PCOS, may also be measured and a ratio of DENND1A. V2 to microRNA may be calculated with a higher ratio (as compared to a healthy control) being indicative of disease when the biological sample is not serum and vice versa when the biological sample is serum. Given that PCOS is the most common cause of anovulatory infertility, the present disclosure provides a quick diagnostic, which may be conducted at a subject's home, that could identify women in need of infertility treatment.

The nucleotide and amino acid sequences of DENND1A. V1 and DENND1A. V2 are presented in US 2015/0087718 and US 2016/0297873, each incorporated herein by reference. For each cDNA sequence presented therein, the disclosure includes the mRNA equivalent of the cDNA, meaning that the disclosure includes each cDNA sequence wherein each T is replaced by U.

As used herein, the term “miRNA” or “microRNA” or “miR” refers to an RNA (or RNA analog) comprising the product of an endogenous, non-coding gene whose precursor RNA transcripts can form small stem-loops from which mature “miRNAs” are cleaved by the endonuclease Dicer. miRNAs are encoded in genes distinct from the mRNAs whose expression they control.

The microRNAs described herein are useful in diagnostic assays for DENND1A. V2 protein, for example, detecting its expression in specific cells, tissues, or serum. Aspects of the disclosure provide a method for diagnosis of a disorder that is positively correlated with expression of DENND1A. V2 mRNA and/or protein in a subject comprising measuring the expression level of one or more microRNAs described herein and comparing the expression level to a reference negative control. The disorder may be PCOS. A reference value may be obtained from samples from healthy, normal patients without the disease. For example, If the measured value of miR-125a-3p (SEQ ID NO: 1), miR-130b-3p (SEQ ID NO: 2), miR-148b-5p (SEQ ID NO: 3), miR-195-5p (SEQ ID NO: 4), and miR-4524a-5p (SEQ ID NO: 5) is lower than the normal reference value when the biological sample is not serum or the same or greater when the biological sample is serum, then it can be concluded that the subject has the disease, and if the measured value is the same or greater than the normal reference value when the biological sample is not serum and lower when the biological sample is serum, then it can be concluded that the subject does not have the disease. If the measured value of miR-501-3p (SEQ ID NO: 6), miR-100-5p (SEQ ID NO: 7), miR-409-5p (SEQ ID NO: 8), miR-1271-5p (SEQ ID NO: 9), miR-1301-3p (SEQ ID NO: 10), miR-99b-5p (SEQ ID NO: 11), miR-127-3p (SEQ ID NO: 12), miR-654-5p (SEQ ID NO: 13), miR-744-5p (SEQ ID NO: 14), miR-1293 (SEQ ID NO: 15), miR-410-3p (SEQ ID NO: 16), miR-502-3p (SEQ ID NO: 17), or miR-494-3p (SEQ ID NO: 18) is greater than the normal reference value when the biological sample is not serum and the same or less than when the biological sample is serum, then it can be concluded that the subject has the disease, and if the measured value is the same or less than the normal reference value when the biological sample is not serum and greater when the biological sample is serum, then it can be concluded that the subject does not have the disease.

In one example, the method as described herein may measures the differential expression of one or more miRNAs as listed above. Thus, in one example, the method may measure the differential expression of at least two, or at least three, or at least four, or at least five, or at least six, or at least seven, or at least eight, or at least nine, or at least 10, or at least 11, or at least 12, or at least 13, or at least 14, or at least 15, or at least 16, or at least 17, or all of the miRNA described herein.

The miRNAs described herein may be used alone or in combination with other markers for prediction of disorders positively correlated with expression of DENND1A. V2 mRNA and/or protein. Exemplary biomarkers include, but are not limited to, measurement of anti-Mullerian hormone, sex hormone binding globulin, testosterone (e.g., total testosterone and/or bioavailable testosterone), HgBA1c, irisin, zinc-α2-glycoprotein, and betatrophin.

In some embodiments, a ratio of expression of a gene associated with PCOS, such as DENND1A. V2, to microRNA expression may be calculated with a higher ratio as compared to a reference ratio obtained from one or more healthy subjects being indicative of disease when the biological sample is not serum and vice versa when the biological sample is serum. If the ratio is the same or lower than the reference ratio when the biological sample is not serum and vice versa when the biological sample is serum, then it can be concluded that the subject does not have the disease.

In certain embodiments, determining a statistically significant increase or decrease of at least 1.5-fold, e.g. 2-3-fold in a sample as compared to a reference is a diagnosis of PCOS or aids in diagnosis of PCOS. In certain embodiments, the increase or decrease relative to a reference is at least 2.0, 3.0 or 4.0-fold, inclusive, and including all digits there between, and to the first decimal place. In one example, the p-values in a statistical test for the change may be lower than 0.05. In one example, the p-values may be calculated using a statistical test known in the art such as a t-test (p-value<0.01). In another example, the statistical test is a t-test (p-value<0.01) that is corrected for false discovery rate (FDR) estimation using Bonferroni-type multiple comparison procedures as known in the art. The reference value may also be a positive control value obtained from patients already diagnosed with the disease or disorder. In this case, if the measured value is the same (or higher/lower depending on the microRNA) than the positive reference value, then it can be concluded that the subject has the disease. In some embodiments, both positive and negative reference values are used in diagnosis. If it is concluded that the subject has the disease, then one can continue with a step or method of treatment as described herein.

Examples of biological samples include, but are not limited to, urine, blood, plasma, serum, and saliva. In some embodiments, the biological sample is a biopsy or surgical specimen of the ovary, endometrium, adipose tissue, or skeletal muscle. In some embodiments, whether an increase or decrease in a microRNA level is observed in a patient having a disorder that is positively correlated with expression of DENND1A. V2 mRNA and/or protein may be dependent on whether a specific compartmental component (i.e., endosomal/exosomal) is examined. For example, a total urine, blood, plasma, serum, or saliva sample may show an increase in miRNAs represented by SEQ ID Nos 1-5 and a decrease in miRNAs represented by SEQ ID Nos 6-18 or vice versa. Fractionation of the sample to a specific compartmental component such as endosomal and/or exosomal compartments may show a decrease in miRNAs represented by SEQ ID Nos 1-5 and an increase in miRNAs represented by SEQ ID Nos 6-18 or vice versa.

In one example, the method is an in vitro method. In one example, the method as described herein may comprise measuring or determining the expression level of at least one microRNA having at least 70%, 80%, or 90% sequence identity, e.g. at least 91%, 92%, 93%, 94%, 95%, 96%, 97%, 98%, or 99% sequence identity, with a miRNA described herein. In one example, the measured miRNAs may have one, two, three or four nucleotide substitutions.

The miRNAs as used in the methods as described herein may be detected using reagents that may be able to hybridize or bind specifically to the miRNA sequences. As used herein, the terms “hybridizing to” and “hybridization” are interchangeably used with the terms “specific for” and “specifically binding” and refer to the sequence specific non-covalent binding interactions with a complementary nucleic acid, for example, interactions between a target nucleic acid sequence and a target specific nucleic acid primer or probe. In one example, a nucleic acid probe, which hybridizes is one which hybridizes with a selectivity of greater than 70%, greater than 80%, greater than 90% or of 100% (i.e. cross hybridization with one of the miRNAs as described herein may occur at less than 30%, less than 20%, less than 10%). As would be understood to a person skilled in the art, a nucleic acid probe, which “hybridizes” to the miRNA as described herein may be determined taking into account the length and composition. In one example, the nucleic acid probes, which hybridize with the any of the miRNAs as described herein may have one, or two, or three mismatched base pairing. In one example, the term “miRNA” as described herein may include miRNAs, which during production, the 3′-end and/or 5′ end of the miRNA may be modified and/or digested. In one example, miRNA that has been modified and/or digested at the 3′-end and/or 5′ end during production is picked up by the assay of the present disclosure. In one example, the miRNA as disclosed herein may include miRNAs which may differ from the sequences as listed in the sequence listing by 3, or 4, or 5 nucleotides.

If the mRNA of a gene associated with PCOS, e.g. DENND1A. V2 is detected, this may be accomplished by a variety of techniques known in the art. Suitable techniques for determining the presence or absence or quantitating mRNA include but are not limited to; hybridization of probes or primers directed to the mRNA, or by using various chip technologies, polynucleotide or oligonucleotide arrays, and combinations thereof. Thus, in various embodiments, probes to the mRNA or a DNA equivalent of it can be arranged and/or fixed on a solid support.

mRNA may be tested directly or may be amplified enzymatically in vitro by, for example, use of the polymerase chain reaction (PCR), Real-Time (RT) PCR, including quantitative real-time (qRT-PCR) PCR analysis, or any other in vitro amplification methods. For amplification reactions, primers can be designed which hybridize to and form a complex with DENND1A Variant 2 mRNA and used to obtain nucleic acid amplification products (i.e., amplicons). Those skilled in the art will recognize how to design suitable primers and perform amplification and/or hybridization reactions in order to carry out various embodiments of the method of the disclosure. In general, the primers should be long enough to be useful in amplification reactions, and generally primers which are at least 12 bases in length are considered suitable for such purposes; but primers as short as 8 bases can be used depending on reaction conditions. The primers/probes used for detecting mRNA can comprise modifications, such as being conjugated to one or more detectable labels; such as fluorophores in the form of a reporter dye and/or a quenching moiety for use in reactions such as real time (RT)-PCR, which allow quantitation of DNA amplified from RNA, wherein the quantitation can be performed over time concurrent with the amplification. In one embodiment, the amplification reaction comprises at least one polynucleotide probe specific for DENND1A Variant 2 mRNA, wherein the probe includes one terminal nucleotide modified to include a fluorescent tag, and the other terminal nucleotide modified to comprise a moiety that quenches fluorescence from the fluorescent tag. For instance, for use in qRT-PCR, such a probe can be designed so that it binds with specificity to a portion of DENNDA1 Variant 2 or its complement, that is between and does not overlap sequences to which two RT-PCR primers hybridize. Using this design, signal from the fluorescent tag will be quenched until the probe is degraded via exonuclease activity of the polymerase during amplification, at which point the fluorescent nucleotide will be separated from the quenching moiety and its signal will be detectable.

If desired, the determination of mRNA and/or protein can be compared to a reference value. The reference to which the mRNA and/or protein levels from the individual can be compared can be any suitable reference, examples of which include but are not limited to samples obtained from individuals who do not have the particular condition for which a diagnosis is sought, such as PCOS. Such references can include matched controls (i.e., matched for age, sex, or other demographics), a standardized curve(s), and/or experimentally designed controls such as known input RNA or protein used to normalize experimental data for qualitative or quantitative determination of the mRNA and/or protein from the sample for mass, molarity, concentration and the like. The reference level may also be depicted graphically as an area on a graph. In certain embodiments, the reference is normal theca cells, which are compared to PCOS theca cells. In another embodiment, the reference is a sample that contains exosomes from an individual who does not have PCOS.

In one example embodiment, the methods as described herein may be provided as a kit. The kit may comprise components necessary for detecting the miRNAs and/or DENND1A. V2 mRNA as described herein. Components may include primers/probes that may hybridizes with the miRNAs as described herein, reagents, containers and/or equipment configured to detect miRNAs and primer/probes as described herein. In one example, the container within the kit may contain a primer/probe set that may detect or determine the level of the miRNAs as described herein that is radiolabelled before use. The kit may comprise containers of substances for reverse transcribing and amplifying one or more miRNAs, such as containers comprising dNTPs (each of the four deoxynucleotides dATP, dCTP, dGTP, and dTTP), buffers, reverse transcriptase and DNA polymerase. The kit may also comprise nucleic acid template(s) for a positive control and/or negative control reaction. The kits may further include any reaction components or buffer necessary for isolation of miRNAs from samples obtained from subjects.

Embodiments of the disclosure relate to the treatment of DENND1A. V2 related disorders. The term “treatment” refers to both therapeutic treatment and prophylactic or preventative measures. Those in need of treatment include those already with the disorder as well as those in whom the disorder is to be prevented. Hence, the mammal to be treated herein may have been diagnosed as having the disorder or may be predisposed or susceptible (e.g. having a genetic predisposition) to the disorder.

The term “mammal” for purposes of treatment refers to any animal classified as a mammal, including humans, domestic and farm animals, and zoo, sports, or pet animals, such as dogs, horses, cats, cows, etc. Preferably, the mammal is human.

PCOS, type II diabetes associated with PCOS, and other disorders of females and males that is the direct result of expression of DENND1A. V2 which results in excess steroid biosynthesis are examples of a disease or disorder positively correlated with expression of DENND1A. V2 mRNA or protein. Affected tissues include, but are not limited to ovarian, vascular smooth muscle, skeletal muscle, adipose tissue, and the endometrium.

Exemplary treatments include, but are not limited to hormonal therapy (such as estrogen, progestin, and gonadotropins), an insulin-sensitizing agent (such as metformin, or pioglitazone), an agent or surgical intervention to increase fertility (such as clomiphene, letrozole, in vitro fertilization, and ovarian “drilling” in which lasers or a thin needle is used to pierce several holes in the surface of the ovary), an antibody or an antigen binding fragment thereof that specifically recognizes DENND1A. V2 protein, and a RNAi containing composition (e.g. shRNA, miRNA, or miRNA mimic such as any of those disclosed herein, or other nucleotide containing compositions), wherein said RNAi composition decreases the expression of DENND1A. V2 or other PCOS gene products such as LHCGR, DENND1A, and RAB5B, among others as described herein.

The term “therapeutically effective amount” refers to an amount of a drug or pharmaceutical composition effective to treat a disease or disorder in a subject. In the case of PCOS or another DENND1A. V2 related disorder, the therapeutically effective amount of the treatment may reduce and/or prevent to some extent one or more of the symptoms associated with the disorder. For example, the amount of measurable testosterone (total and free), androstenedione, and dehydroepiandrosterone sulfate (DHEAS) in a biological sample obtained from a subject being treated as described herein (e.g., a blood sample) may decrease, e.g. at least about 10, 20, 30, 40, 50, 60, 70, 80 or 90% or more; and/or other symptoms may be ameliorated, e.g. ovulatory cycles will return, ovarian morphology may normalize, including disappearance of multiple follicular cysts, and hyperinsulinemia and insulin resistance may be diminished.

In one aspect, the present disclosure comprises one or more isolated and/or recombinantly or otherwise synthesized (e.g., chemically synthesized) binding partners which specifically recognize the DENND1A. V2 protein, but do not specifically recognize DENND1A. V1 protein for the treatment of a DENND1A. V2-related disorder. Exemplary binding partners are disclosed in US 2015/0087718 and US 2016/0297873, each incorporated herein by reference.

Such binding partners include antibodies and antigen binding fragments thereof that can specifically bind to the unique C-terminus of the DENND1A. V2 protein, such as Fab fragments, Fab′ fragments, F(ab′)2 fragments, Fd fragments, Fv fragments, scFv fragments, aptamers, diabodies and combinations thereof. Single-chain Fv or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. The antibody and antigen binding fragments thereof are directed to one or more epitopes in the unique 33 amino acid C-terminus of the DENND1A. V2 protein as set forth in US 2015/0087718 and US 2016/0297873, each incorporated herein by reference.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

Antibodies and methods for preparation of antibodies are well-known in the art. Details of methods of antibody generation and screening of generated antibodies for substantially specific binding to an antigen are described in standard references such as E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; and B. K. C. Lo, Antibody Engineering: Methods and Protocols, Methods in Molecular Biology, Humana Press, 2003.

An antibody or antigen-binding fragment of the disclosure specifically recognizes or binds a target epitope when it displays no more than 5% binding to other epitopes, and preferably displays no measurable binding to other epitopes, for example when measured by an enzyme-linked immunosorbent assay. An antibody or antigen-binding fragment does not specifically recognize or bind a target epitope if it displays more than 5% binding to other epitopes. Antibodies or antigen-binding fragments that bind non-specifically may still bind selectively, for example, an antibody may bind the target epitope as well as display non-specific binding to a few other epitopes.

Any antibody produced by a non-human mammal derived hybridoma can be modified to provide a chimeric, or partially or fully humanized form; and the present disclosure includes such modifications. In general, “humanized” forms of non-human (e.g., mice) antibodies are chimeric antibodies that contain a minimal sequence derived from the non-human antibody. Humanized antibodies are essentially human immunoglobulins (also called the “recipient” antibody) in which residues from a hypervariable region of the recipient are replaced by residues from a hypervariable region of a non-human species (also called a “donor” antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also can comprise at least a portion of an immunoglobulin constant region (Fc), typically that of a human immunoglobulin. For further details, see Jones et al., [27]; Riechmann et al., [28]; and Presta, [29].

Methods for humanizing non-human antibodies are well known in the art. Humanization of an antibody produced according to the present disclosure can be essentially performed following the method of Winter and co-workers by substituting mouse CDR sequences for the corresponding sequences of a human antibody, Jones et al [27]; Riechmann et al., [28]; and Verhoeyen et al., [30].

In another embodiment, the disclosure includes an antigen-binding or variable region fragment of an antibody described herein. Examples of suitable antibody fragments include Fab, Fab′, F(ab′)₂ and Fv fragments. Various techniques have been developed for the production of antibody fragments. Traditionally, these fragments were derived via proteolytic digestion of intact antibodies. However, these fragments can now be produced directly by recombinant host cells. For example, the antibody fragments can be isolated from antibody phage libraries as described below. Alternatively, Fab′-SH fragments can be directly recovered from bacterial expression systems and chemically coupled to form F(ab′)2 fragments.

In one embodiment, a library of proteins that have antigen binding regions can be screened to identify candidates that can be modified for therapeutic purposes according to this disclosure. For example, a phage display or other antibody library can be screened against a DENND1A. V2 C-terminal polypeptide described herein, and DENND1A. V2 C-terminal specific binding proteins can be identified. The CDR sequences of the light and heavy chains encoded by the phage DNA, or any other DNA that encodes the antigen binding regions, can be determined using techniques known to those skilled in the art. The CDR sequences can then be cloned into expression vectors to produce DENND1A. V2 C-terminal specific antibodies or DENND1A. V2 C-terminal specific fragments thereof as described above. Thus, in embodiments, the DENND1A. V2 C-terminal specific antibodies or DENND1A. V2 C-terminal specific fragments thereof as described above will be distinct from polypeptides in the library. In an embodiment, the DENND1A. V2 C-terminal specific antibodies or DENND1A. V2 C-terminal specific fragments thereof do not contain any phage/phagemid protein, including but not necessarily limited to bacteriophage coat protein(s). Newer approaches for generating antibodies or antigen binding fragments such as the non-limiting example of camel nanobodies are contemplated herein.

The disclosure also provides isolated nucleic acids encoding the anti-DENND1A. V2 antibodies, vectors and host cells comprising the nucleic acids, and recombinant techniques for treating DENND1A. V2-related disorders.

In further embodiments, the treatments described herein such as antibodies or antigen binding fragments may be administered by any suitable means, including, but not limited to; subcutaneous, parenteral, vaginally, intraperitoneal, intrapulmonary, and intranasal. Parenteral infusions include intravenous, intramuscular, intraarterial, intraperitoneal, intralymphatic or subcutaneous administration. In addition, the treatment may be administered by pulse infusion, e.g., with declining doses. Methods of oral administration are also contemplated. Slow-release formulations may be used with modifications such as pegylation to extend the half-life of the pharmaceutical composition.

For the prevention or treatment of disease, the appropriate dosage will depend on the type of disease to be treated, the severity and course of the disease, whether the treatment is administered for preventive and/or therapeutic purposes, previous therapy, the patient's clinical history and response to the antibody, and the discretion of the attending physician. The therapy is suitably administered to the patient at one time or over a series of treatments. For example, depending on the type and severity of the disease, about 1 μg/kg to 15 mg/kg (for example, 0.1-20 mg/kg) of antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. The preferred dosage of the antibody will be in the range from about 0.5 mg/kg to about 10 mg/kg. Thus, one or more doses of about 0.5 mg/kg, 2 mg/kg, 4 mg/kg or 10 mg/kg (or any combination thereof) may be administered to the patient. Such doses may be administered intermittently, for example, every week or every three weeks. An initial higher loading dose, followed by one or more lower doses may be administered. An exemplary dosing regimen comprises administering an initial loading dose of about 4 mg/kg, followed by a weekly maintenance dose of about 2 mg/kg of the anti-DENND1A. V2 antibody. However, other dosage regimens may be useful. The progress of this therapy is easily monitored by conventional techniques and assays. Aside from administration of the antibody protein to the patient, the present application contemplates administration of the antibody by gene therapy. Such administration of nucleic acid encoding the antibody is encompassed by the expression “administering a therapeutically effective amount of an antibody”.

There are two major approaches to delivering nucleic acid (optionally contained in a vector) into the patient's cells, in vivo and ex vivo. For in vivo delivery the nucleic acid is injected directly into the patient, e.g. into the bloodstream or at the site where the antibody is required. For ex vivo treatment, the patient's cells are removed, the nucleic acid is introduced into these isolated cells and the modified cells are administered to the patient either directly or, for example, encapsulated within porous membranes which are re-implanted into the patient. There are a variety of techniques available for introducing nucleic acids into viable cells. The techniques vary depending upon whether the nucleic acid is transferred into cultured cells in vitro or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

Exemplary in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Choi, for example). In some situations, it is desirable to provide the nucleic acid source with an agent that targets the target cells, such as an antibody specific for a cell surface membrane protein of the target cell, a ligand for a receptor on the target cell, etc. Where liposomes are employed, proteins which bind to a cell surface membrane protein associated with endocytosis may be used for targeting and/or to facilitate uptake; for example capsid proteins or fragments thereof tropic for a particular cell type, antibodies for proteins which undergo internalization in cycling, and proteins that target intracellular localization and enhance intracellular half-life.

In some embodiments, the targeted cells are theca cells and contacting the theca cells with the antibodies or antigen binding fragments described herein decreases androgen and/or progesterone biosynthesis in the theca cells and/or decreases CYP17A1 and/or CYP11A1 gene expression, as well as CYP17 and CYP11A1 mRNA and/or protein in the cells. These decreases reduce or prevent symptoms associated with overexpression of CYP17A1 and/or CYP11A1 gene regulation and CYP17 and/or CYP11A1 mRNA and/or protein, for example, excess androgen production. Exemplary symptoms of excess androgen production which may be lessened or resolved include but are not limited to: anovulation associated with hyperandrogenemia, hirsutism, ovarian morphology, and infertility. In some embodiments, the treatments described herein reduce or prevent other phenotypes associated with PCOS, for example abnormal insulin signaling, insulin resistance in adipose, skeletal muscle, and endometrial tissue, abnormal FSH signaling in granulosa cells, and combinations thereof.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention will be further illustrated by the following examples. However, these examples and figures should not be interpreted in any way as limiting the scope of the present invention.

### Example 1

### Materials and Methods

Cell Culture. Human theca interna tissue was isolated from the ovaries of age-matched, normal cycling women (n=7) and PCOS women (N=7) using a protocol approved by the Institutional Review Board of The Pennsylvania State University College of Medicine as previously described^{23, 24, 61, 62}. As a standard of care, oophorectomies were performed during the luteal phase of the cycle. Theca cells from normal cycling and PCOS follicles were isolated and grown as we have previously reported in detail^{63, 64}. The theca cell preparations used in these studies have been described and characterized previously^{23, 24, 29, 30, 63-67}. The steroidogenic phenotypes of the normal and PCOS theca cells have been reported to result from the inherent properties of the cells, rather than the cycle phase at the time that they were isolated^{24, 65, 68}. PCOS and normal ovarian tissue came from age-matched women, 38-40 years old. The diagnosis of PCOS was made according to National Institutes of Health (NIH) consensus guidelines^{4, 69}, which include hyperandrogenemia, oligoovulation, polycystic ovaries, and the exclusion of 21α-hydroxylase deficiency, Cushing's syndrome, and hyperprolactinemia. All of the PCOS theca cell preparations studied came from ovaries of women with fewer than six menses per year and elevated serum total testosterone or bioavailable testosterone levels^{24, 65}. Each of the PCOS ovaries contained multiple subcortical follicles of less than 10 mm in diameter. The control (normal) theca cell preparations came from ovaries of fertile women with normal menstrual histories, menstrual cycles of 21-35 days, and no clinical signs of hyperandrogenism. Neither PCOS nor normal subjects were receiving hormonal medications at the time of surgery. Indications for surgery were dysfunctional uterine bleeding, endometrial cancer, and pelvic pain. Experiments comparing PCOS and normal theca were performed using fourth-passage (31-38 population doublings) theca cells isolated from individual size-matched follicles obtained from age-matched subjects, in the absence of in vivo stimulation. The use of fourth-passage cells allowed us to perform multiple experiments from the same patient population and were propagated from frozen stocks of primary and second passage cells in the media described above. As such, the passaged normal and PCOS theca cells are not cell lines. The passage conditions and split ratios for all normal and PCOS cells were identical. Fourth passage normal and PCOS theca cells for these studies, were grown until sub-confluent, and were treated with and without 20 μM forskolin for 16 hours, in defined serum free media^{61, 62}.

For experiments utilizing the adrenocortical H295R cell line that were obtained from ATCC⁷⁰, we followed ATCC's recommended media and culture conditions. H295R cells were grown to subconfluency and treated with and without 20 μM forskolin in the defined serum free media used routinely for normal and PCOS theca cells^{61, 62}.

MicroRNA global deep sequencing in normal and PCOS theca cells. MicroRNA deep sequencing was performed using Illumina technology (by LC Sciences, lcsciences.com) on theca cell RNA from normal cycling women (n=4) and women with PCOS (n=4) each treated with (F) and without (C) 20 μM forskolin. Total RNA was extracted using TRIzol reagents. Small RNA library prep, cDNA deep sequencing and initial data analysis including alignment of raw reads to miR-Base (22 release) and normalization of count data was performed by LC Sciences. The log₂ normalized expression levels for each miRNA were used for statistical analyses after filtering for human miRNA and can be accessed at: ncbi.nlm.nih.gov/geo/query/acc.cgi?token=cdyxguqsljaltit&acc=GSE92862.

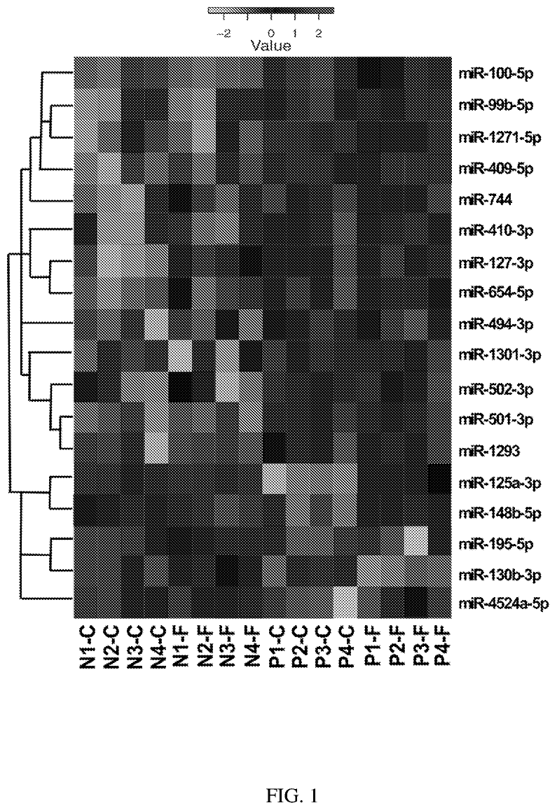

To perform miRNA discovery sequencing analysis for each microRNA, a one way analysis of variance (ANOVA) model was fit to the log₂ normalized expression levels to identify miRNA differentially expressed among the four groups: Normal Untreated Control (Normal-C), Normal Forskolin Treated (Normal-F), PCOS Untreated Control (PCOS-C) and PCOS Forskolin Treated (PCOS-F). After fitting the ANOVA models for each miRNA, linear contrasts were used to test for differences between the following group comparisons: Normal-C vs. Normal-F; PCOS-C vs. PCOS-F; Normal-C vs. PCOS-C; Normal-F vs. PCOS-F and Untreated (Normal-C and PCOS-C) vs. Treated (Normal-F and PCOS-F). Meta-analysis was performed on miRNA common between and being significant (P<0.05) in both group comparisons (Table 1); Normal-C vs. PCOS-C, and Normal-F vs PCOS-F. Their individual P values were combined using Fisher's method for combining P values to get an overall comparison of miRNA expression between PCOS and normal theca cells. Subsequently, the combined P values were used to obtain the false discovery rate (FDR) using the Benjamini and Hochberg method. MicroRNAs having a statistically significant expression difference between both pair-wise comparisons (Normal-C vs. PCOS-C and Normal-F vs. PCOS-F), a meta-analysis combined P<0.05 and FDR <0.05 were considered significant. All statistical analyses for the microRNA profiling were performed in R package for Statistical Computing (R-project, r-project.org/). A heatmap for this data (FIG. 1) was prepared by LC Sciences, using the mean expression data from 4 preparations of normal (N1, N2, N3, N4) and PCOS theca cells (P1, P2, P3, P4), treated in the absence (C) and presence of 20 μM forskolin (F).

TABLE 1
Differentially expressed micoRNAs in normal and PCOS theca cells. MicroRNA deep sequencing on theca cell RNA from
normal cycling women (n = 4) and women with PCOS (n = 4) each treated with and without 20 μM forskolin. For microRNA
discovery sequencing analysis, a one way analysis of variance (ANOVA) model was fit to the log₂ normalized expression
levels to identify miRNA differentially expressed among the four groups: Normal Untreated Control (Normal-C), Normal
Forskolin Treated (Normal-F), PCOS Untreated Control (PCOS-C) and PCOS Forskolin Treated (PCOS-F). We identified
18 miRs that were observed to be differentially expressed between Normal and PCOS theca cells. 13 of these 18 miRs were
upregulated (>1.0), and 5 miRs were down-regulated (<1.0) in PCOS theca cells compared to normal cells (Fisher's combined
P value <0.05, and FDR <0.05 are considered significant). For each of these 18 microRNAs we present mean expression
values for normal and PCOS cells under control (C) and forskolin (F) stimulated conditions, as well as the ratio of mean
expression for each and statistical significance.

| | Normal | PCOS | PCOS/ | P value | Normal | PCOS | PCOS/ | P value | | |
|---|---|---|---|---|---|---|---|---|---|---|
| mRNA | C | C | Normal | P vs N | F | F | Normal | P vs N | Meta | Meta |
| (hsa) | Mean | Mean | (C) | (C) | Mean | Mean | (F) | (F) | P value | FDR |
| miR-501-3p | 92.43 | 583.10 | 6.31 | 2.13E−06 | 97.84 | 544.31 | 5.56 | 5.20e−06 | 2.90E−10 | 5.29E−07 |
| miR-100-5p | 40687.07 | 198820.24 | 4.89 | 4.74E−06 | 36212.81 | 141097.22 | 3.90 | 2.83e−05 | 3.19E−09 | 2.43E−06 |
| miR-409-5p | 87.38 | 810.37 | 9.27 | 4.66E−06 | 146.62 | 954.57 | 6.51 | 3.65e−05 | 4.00E−09 | 2.43E−06 |
| miR-125a-3p | 391.08 | 271.26 | 0.69 | 9.19E−06 | 448.17 | 340.06 | 0.76 | 1.17e−04 | 2.32E−08 | 1.06E−05 |
| miR-1271-5p | 47.68 | 354.57 | 7.44 | 8.83E−05 | 45.34 | 254.33 | 5.61 | 3.70e−04 | 5.95E−07 | 2.17E−04 |
| miR-1301-3p | 22.93 | 61.96 | 2.70 | 1.04E−03 | 21.05 | 64.88 | 3.08 | 2.87e−04 | 4.79E−06 | 1.45E−03 |
| miR-130b-3p | 1057.38 | 644.83 | 0.61 | 3.63E−04 | 995.84 | 714.19 | 0.72 | 6.70e−03 | 3.39E−05 | 8.23E−03 |
| miR-99b-5p | 15624.57 | 43641.17 | 2.79 | 7.42E−04 | 15478.35 | 36681.54 | 2.37 | 3.51e−03 | 3.61E−05 | 8.23E−03 |
| miR-127-3p | 24794.80 | 53267.35 | 2.15 | 1.72E−04 | 38179.62 | 54137.86 | 1.42 | 2.86e−02 | 6.45E−05 | 1.31E−02 |
| miR-148b-5p | 190.69 | 132.42 | 0.69 | 1.55E−03 | 268.62 | 200.59 | 0.75 | 6.33e−03 | 1.23E−04 | 2.18E−02 |
| miR-654-5p | 322.62 | 779.03 | 2.41 | 1.72E−04 | 409.07 | 561.48 | 1.37 | 6.35e−02 | 1.35E−04 | 2.18E−02 |
| miR-195-5p | 175.08 | 80.40 | 0.46 | 8.74E−04 | 122.19 | 75.29 | 0.62 | 1.33e−02 | 1.44E−04 | 2.18E−02 |
| miR-744-5p | 887.00 | 1317.24 | 1.48 | 2.10E−03 | 1012.80 | 1374.08 | 1.36 | 1.00e−02 | 2.48E−04 | 3.29E−02 |
| mir-1293-p3 | 6.23 | 12.78 | 2.05 | 8.56E−03 | 6.84 | 15.82 | 2.31 | 2.80e−03 | 2.78E−04 | 3.29E−02 |
| miR-410-3p | 3016.03 | 5096.84 | 1.69 | 1.30E−03 | 3167.66 | 4458.61 | 1.41 | 1.92e−02 | 2.90E−04 | 3.29E−02 |
| miR-4524a-5p | 18.37 | 5.74 | 0.31 | 9.42E−04 | 13.80 | 6.82 | 0.50 | 2.67e−02 | 2.91E−04 | 3.29E−02 |
| miR-502-3p | 34.94 | 62.00 | 1.77 | 1.52E−02 | 30.01 | 66.75 | 2.22 | 1.75e−03 | 3.07E−04 | 3.29E−02 |
| miR-494-3p | 102.78 | 260.35 | 2.53 | 5.50E−03 | 130.48 | 339.02 | 2.60 | 5.95e−03 | 3.71E−04 | 3.76E−02 |

MicroRNA target identification. The microRNA target prediction filter in Qiagen's Ingenuity® Pathway Analysis tool (IPA®, QIAGEN Redwood City, qiagen.com/ingenuity), miR-base (mirbase.org), and miR-TargetMiner (isical.ac.in/˜bioinfo_miu/targetminer20.htm) were used to identify potential target genes for the 18 miRNAs (Table 2) that were differentially expressed between PCOS and normal theca cells (FDR <0.05). MicroRNA-target gene relationships having a confidence level of “Experimentally Observed” or “High-Predicted” defined by this analysis were primarily considered to be significant.

TABLE 2
MicroRNA target identification identified miR-130b-3p as a candidate
mediating DENND1A.V2 expression in normal and PCOS theca cells.
Target identification analyses (miR-Base, IPA target filter, and miR-
TargetMiner) predicted that 5 of the 18 differentially expressed
microRNAs, targeted 9 of the 18 PCOS GWAS loci that have been
validated in several ethnic populations. As indicated in bold, miR-130b-
3p was found to target the PCOS GWAS loci, including DENND1A,
ZNF217, RAB5B, LHCGR, and ERBB3. Of particular interest to these
studies, miR-130b-3p was observed to target DENND1A***, a
functional PCOS GWAS candidate shown to mediate excess androgen
production in PCOS theca cells.

| ID | Targeted |
|---|---|
| (hsa) | Gene |
| miR-130b-3p | DENND1A*** |
| miR-130b-3p | ZNF217 |
| miR-130b-3p | R4258 |
| miR-130b-3p | LHCGR |
| miR-130b-3p | ERBB3 |
| miR-130b-3p | KCNA4 |
| miR-195-5p | INSR |
| miR-195-5p | YAP1 |
| miR-195-5p | HMGA2 |
| miR-654-5p | GATA4 |
| miR-1271-5p | ERBB3 |

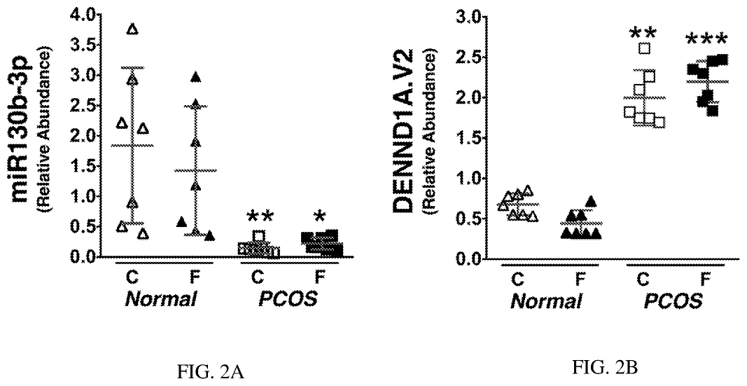

Quantitative RT-PCR based analysis of miRNA expression. The expression profile of miR-130b-3p in normal and PCOS theca cells was validated in vitro using the TaqMan® MicroRNA Assay (ThermoFisher, Pleasanton, Calif.) following the manufacturer's instructions. For the miRNA-qPCR assays, total RNA was extracted from the established theca cell cultures (Normal: n=7 and PCOS: n=7; treated with and without forskolin) using Tri-reagent (Millipore-Sigma, St. Louis, Mo.). Briefly, 10 ng RNA was reverse transcribed using the target (miRNA) specific stem-loop RT primer and the TaqMan® MicroRNA reverse transcription kit (ThermoFisher, Pleasanton, Calif.). The cDNA was then amplified by real-time qRT-PCR using target specific TaqMan® primer-probe mix. The qRT-PCR was performed in triplicate per sample and U6 small nuclear 1 (RNU6-1) was utilized for normalization of the miR-130b-3p qRT-PCR expression data. The mean expression value for each miRNA was divided by the mean RNU6-1 expression value to normalize each sample (FIG. 2).

Quantitative real-time qRT-PCR analyses of DENND1A. V1, DENND1A. V2, and CYP17A. Quantitation of DENND1A. V1, DENND1A. V2 and CYP17A1 mRNA abundance was determined using the Single Step Brilliant III Ultra Fast qRT-PCR kit (Agilent, Santa Clara, Calif.), using primer and probe sets as we have previously described in detail⁶¹. The gene specific one step PCR was carried out in duplicate for each mRNA sample and for a series of dilutions in an Agilent AriaMx® Real-Time PCR System (Santa Clara, Calif.) according to manufacturer's instructions for this instrument as previously described⁶¹. TATA Binding Protein (TBP) mRNA used for normalization and the mean expression value for each mRNA was divided by the mean TBP expression value to normalize each sample⁶¹.

Quantitation of dehydroepiandrosterone (DHEA). ELISA for DHEA was performed on cell culture media collected from the identical 7 normal and 7 PCOS theca cell preparations (treated with and without 20 μM forskolin) that were utilized in parallel studies to examine miR-130b-3p, DENND1A. V2, DENND1A. V1, and CYP17A1 mRNA accumulation. The DHEA ELISAs were performed without organic solvent extraction using kits from DRG International, Inc. (Springfield, N.J.) as described by the manufacturer's protocol, and normalized by cell count as previously described^{24, 61}. The specificity and cross-reactivity of the specific DHEA ELISA antibody (RRID:AB_2800459), was determined by DRG International Inc., (Springfield, N.J.). DHEA ELISA antibody, displayed 100% cross reactivity with DHEA, and the following cross-reactivity with 17α-hydroxy-pregnenolone, 0.072%; androsterone, 0.056%; desoxycorticosterone, 0.052%; progesterone, 0.023% and pregnenolone, 0.013% and less than 0.01% for 11-desoxycortisol, corticosterone, DHEAS, T, and 5α-dyhydrotestosterone.

Analysis of DENND1.V2 and DENND1A. V1 mRNA accumulation in response to hsa-miR-130b-3p mimic in H295R cells. The highly androgenic human Adrenocortical H295R cell line was utilized for our miRNA mimic studies⁷¹, because of the limitations of using human theca cells described below. Human Adrenocortical H295R cells were transfected with 75 pM mirVana hsa-miR-130b-3p mimic or negative control-1 mimic (Life Technologies, Waltham, Mass.) using RNAiMAX transfection reagents (Invitrogen, Waltham, Mass.) using the manufacturer's protocol, in Opti-MEM serum free medium (Thermo Fisher, Waltham, Mass.). Eight hours following transfection the cells were treated with and without 20 μM forskolin, and total RNA from the cells were harvested 48 h thereafter. The effects of miR-130b-3p mimic on DENND1A. V2, DEND1A. V1 and CYP17A1 mRNA accumulation were quantified as described above. The human adrenocortical H295R cell line was utilized for these miR-mimic studies instead of normal or PCOS theca cells, because the reagents/conditions required for miR-mimic transfection were toxic to passaged theca cells when the experiments were performed. The H295R cell line also has a relatively high transfection efficiency (>25%), compared to early passaged human theca cells (<10-15%), under the most moderate of conditions. As we have previously reported, forced DENND1A. V2 mRNA and protein expression in the Adrenocortical H295R cell line via adenoviral infection increased CYP17A1 gene expression and DHEA production⁷¹, concordant with results observed in normal theca cells⁶¹.

Western blot analysis of DENND1.V2 and DENND1A. V1 protein in miR-130b-3p mimic transfected H295R cells. The Adrenocortical H295R cell line was grown until subconfluent and transfected with hsa-miR-130b-3p and negative control mimic (Neg-mimic) and treated with or without forskolin as described above. 48 hours thereafter, the cells were harvested in RIPA, and Western blot analyses were performed using 30 μg protein per lane as we have previously described⁶¹. A N-terminal Sigma antibody for intra-peptide sequence of DENND1A (RRID:AB_2800456)⁷², which binds both DENND1A. V1 (119 kD) and DENND1A. V2 (62 kD), and a rabbit polyclonal specific for the unique 32 amino acid C-terminal sequence of DENND1A. V2 (RRID:AB_2800456), were used as primary antibodies, as we have previously reported⁶¹. DENND1A. V2 and DENND1A. V1 protein was visualized using ECL (Rockland Immunochemicals), and quantitated using a ProteinSimple® FluorChem® R (Pottstown, Pa.). Both DENND1A. V2 and DENND1A. V1 protein were normalized using total mTOR antibody⁷³, which is not significantly different in normal and PCOS theca cells, nor regulated by forskolin treatment⁶¹. These experiments were repeated ˜4 times, and the data presented is presented as cumulative results.

IPA core network pathway analysis. The miRNA target prediction filter in Qiagen's Ingenuity® Pathway Analysis tool (IPA®, QIAGEN Redwood City, qiagen.com/ingenuity) was used to identify potential target genes for miR-130b-3p. Core pathway and network analyses using the “Grow” and “Build” network functions available in IPA utilize drop-down analyses tools and options designed within the IPA analyses tool. This analysis takes into account any known or predicted connections present in the Ingenuity Knowledge base, which took into account the set parameters defined to be associated with PCOS and miR-130b-3p in an extensive IPA library of previously reported gene/protein/function interactions.

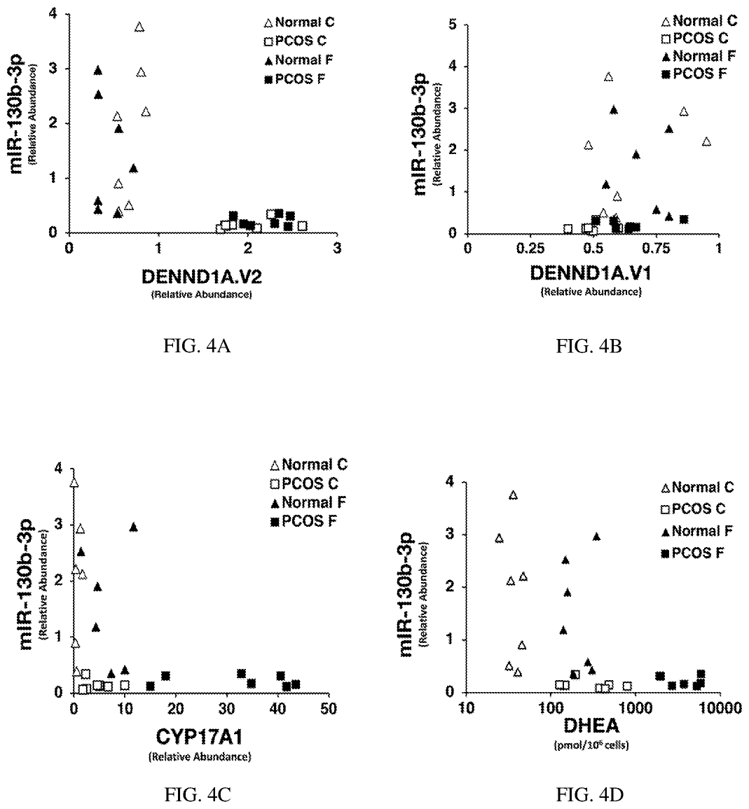

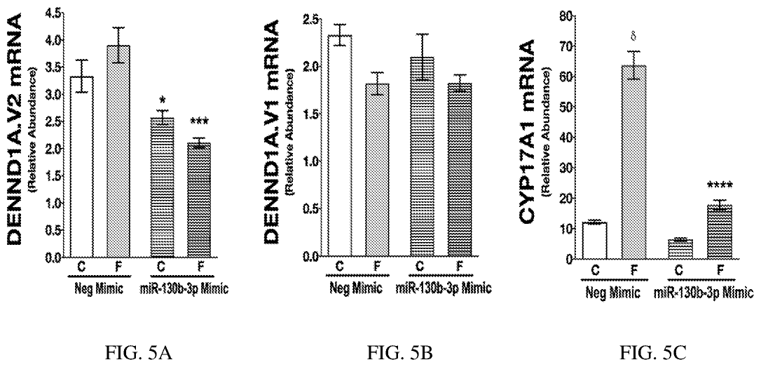

Statistical Analysis. For comparisons of miR-130b-3p, DENND1A. V2, DENND1A. V1, and CYP17A mRNA in normal and PCOS theca cells, 1-way ANOVA was performed to test for differences, and P values were determined by the Bonferroni method for multiple comparisons, between groups Normal-C vs. PCOS-C and Normal-F vs. PCOS-F, and P<0.05 were considered statistically significant (FIGS. 2 and 5). Correlations were determined from experiments where miR-130b-3p, DENND1A. V2, DENND1A. V1 and CYP17A1 mRNA, and steroid (DHEA) were examined from multiple individual patients under treated and untreated conditions (FIG. 4). Spearman's nonparametric correlation coefficient (p) was calculated to estimate the degree of correlation between all pairwise comparisons (p was considered significant when P<0.05), using JMP (FIG. 4). The mRNA and Western analyses data for DENND1A. V2 and DENND1A. V1 following miR-130b-3p mimic transfection were analyzed by 1-way ANOVA, and P values were determined by the Bonferroni method for multiple comparisons (FIGS. 5-7).

### Results

Expression profiling of differentially expressed microRNAs in theca cells isolated from normal cycling and PCOS women. For microRNA profiling, small RNA deep-sequencing (miR-seq) was performed using libraries prepared from RNA isolated from 4^{th} passage theca cells, from 4 individual normal cycling and 4 individual PCOS women treated for 16 h under control non-stimulated (C) and 20 μM forskolin-stimulated (F) conditions, by LC Sciences (see Materials and Methods). A total of 1,823 miRNA were identified from small RNA deep sequencing to be differentially expressed in normal and PCOS theca cells. After meta-analysis of the Normal-C vs. PCOS-C and Normal-F vs. PCOS-F pairwise comparisons (described in detail in Materials and Methods), 18 miRNAs were found to have the highest statistical differences in expression, between theca cells obtained from 4 normal cycling and 4 PCOS women (Fisher's combined P<0.05) with a FDR <0.05 (Table 1). For each of these 18 microRNAs in Table 1, we present the mean expression values for normal and PCOS cells under control (C) and forskolin (F) stimulated conditions, as well as the ratio of mean expression for each and statistical significance. Of the most significant 18 microRNAs observed to be differentially expressed, 13 had increased expression and 5 had decreased expression in PCOS theca cells as compared to normal theca cells.

As presented in FIG. 1, a heat-map with hierarchical clustering was prepared using the global normalization expression data and the relative copy numbers of the 18 microRNAs found to have the highest statistical differences between theca cells propagated from 4 normal cycling and PCOS women (P<0.001) treated in the absence (C) and presence (F) of 20 μM forskolin. As shown in FIG. 1, hierarchical clustering of the 18 differentially expressed microRNAs (indicated on the left), suggest that there are 2 families of related microRNAs that mediate microRNA expression in normal and PCOS theca cells. Each of the individual groups or families appears to mediate increased expression in PCOS theca cells in contrast to increased expression in normal theca cells.

Target analyses predict that several of differentially expressed miRNA in normal and PCOS theca cells target PCOS candidate genes, including DENND1A. The 18 miRNA (listed in Table 1 and FIG. 2) were used for bioinformatics analysis including target prediction in IPA. The miRNA target prediction filter in IPA was selected to identify the validated and predicted PCOS GWAS target genes of the differentially expressed miRNA. As shown in Table 2, target prediction revealed that 4 of the 18 (˜22%) of the miRNA that are differentially expressed between normal and PCOS theca cells (i.e., miR-130b-3p, miR-195-5p, and miR-654-5p, and miR-1271-5p) were predicted to target 10 of the 22 (45%) of the PCOS GWAS candidate genes (i.e., DENND1A, INSR, LHCGR, RAB5B, ZNF217, YAP1, GATA4, HMGA2, ERBB3, and KCNA4), identified in Han-Chinese^{52, 53} and European populations^{54, 55} following both IPA analyses, miR-Base, and mIR-Target Miner Data Base analyses. A total of 8366 additional interactions were also identified by IPA. Noticeable in Table 2, miR-130b-3p was predicted to have target interactions with PCOS GWAS candidates DENND1A, ZNF217, RAB5B, LHCGR, ERBB3, and KCNA4. Of particular interest, miR-130b-3p was observed to target DENND1A, which we previously identified as a functional GWAS candidate mediating excess androgen production in PCOS theca cells⁶¹. Notably, LHCGR, DENND1A variant 2 and RABSB co-localize in theca cells and DENND1A variant 2 and RABSB are translocated into the theca cell nucleus in polycystic ovary syndrome theca cells (Colocalization of Polycystic Ovary Syndrome Candidate Gene Products in Theca Cells Suggests Novel Signaling Pathways. Kulkarni R, Teves M E, Han A X, McAllister J M, Strauss J F 3rd. J Endocr Soc. 2019 Sep. 16; 3(12):2204-2223. doi: 10.1210/js.2019-00169. eCollection 2019 Dec. 1).

miR-130b-3p expression is decreased and DENND1A. V2 expression is increased in PCOS theca cells as compared to normal theca cells. Based on target analyses that predicted that miR-130b-3p targets DENND1A. V2 (Table 2) we used qRT-PCR to examine the differential expression of both miR-130b-3p and DENND1A. V2 in a larger population of theca cells isolated from 7 different normal cycling and 7 different PCOS women treated with either 20 μM forskolin or under control non-stimulated conditions for 24 h, as described in Materials and Methods. As shown in FIG. 2A, miR-130b-3p was observed to be significantly decreased in PCOS theca cells under both control (P<0.01) and forskolin-treated (P<0.05) conditions compared to normal theca cells. As shown in FIG. 2B, an examination of DENND1A. V2 mRNA expression in the identical 7 normal and 7 PCOS mRNA samples utilized to examine miR-130b-3p, demonstrated a statistical increase in DENND1A. V2 mRNA abundance in PCOS theca cells as compared to normal theca cells. Forskolin treatment had no effect on miR-130b-3p or DENND1A. V2 expression in normal or PCOS theca cells.

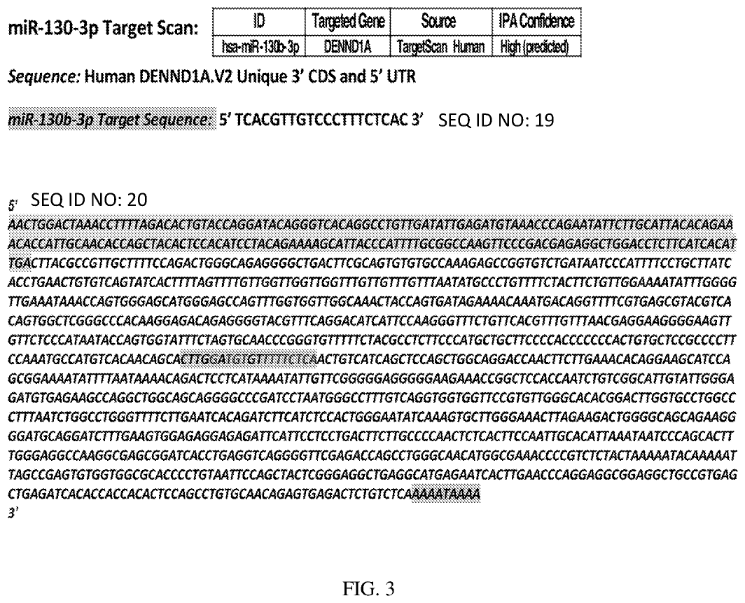

MicroRNA target analysis provided evidence that miR-130b-3p targets the DENND1A. V2 3′UTR. In our initial analysis, miR-Target Minor predicted a miR-130b-3p target seed sequence at approximately 330 bp in the 3′UTR of DENND1A. However, the DENND1A(.V2) 3′UTR sequence used in this first approach was not found to be homologous to the DENND1A. V2 splice variant and associated 3′UTR. Using a more specific approach using the miR-130b-3p target sequence and correct DENND1A. V2 C-terminal CDS and 3′UTR we were able to identify the mIR-130b-3p seed sequence approximately 527 bp past the DENND1A. V2 CDS stop codon (ATTGA), using a combination of TargetScan, IPA Confidence, and Target-Miner analyses (FIG. 3). The DENND1A. V2 polyadenylation signal AAAATAAAA is highlighted approximately 1221 bp 3′ of the DENND1A. V2 CDS stop codon.

Decreased miR-130b-3p expression is correlated with increased DENND1A. V2 and CYP17A1 mRNA expression and androgen biosynthesis in PCOS theca cells. Based on the miR130b-3p DENND1A. V2 3′UTR target analyses (FIG. 3), and data demonstrating decreased miR-130b-3p expression (FIG. 2A) and increased DENND1A. V2 mRNA expression (FIG. 2B) in PCOS theca cells (FIG. 2); experiments were performed to evaluate the extent which miR-130b-3p expression is correlated with the expression patterns of truncated DENND1A. V2, full length DENND1A. V1 and CYP17A1 mRNAs as well as DHEA accumulation. Real-time PCR for DENND1A. V1 and CYP17A1 performed using remaining RNA harvested from the same 7 individual normal and 7 PCOS theca cell preparations used for the experiments and results presented in FIG. 2, which were treated in the presence (F) and absence (C) of 20 μM forskolin for 16 hr. Parallel experiments were also performed to examine the relationship between miR-130b-3p and DHEA biosynthesis in the identical 7 normal and 7 PCOS theca cell preparations. In FIG. 4A, the comparison of DENND1A. V2 mRNA expression with miR-130-3p for each individual preparation of normal and PCOS theca cells, established that there was a significant negative correlation between miR-130b-3p and DENND1A. V2 mRNA. We also observed an unexpected positive relationship between miR-130b-3p and DENND1A. V1 mRNA expression, as shown in FIG. 4B. There was also a significant negative correlation between miR-130b-3p and CYP17A1 mRNA abundance (FIG. 4C), and a significant negative correlation between miR-130b-3p levels and DHEA accumulation was observed in PCOS theca cells (FIG. 4D). These data support the notion that decreased miR-130b-3p expression in PCOS theca cells is concomitant with augmented DENND1A. V2 and CYP17A1 mRNA expression and down-stream increases in androgen biosynthesis in PCOS theca cells.

Overexpression of a miR-130b-3p mimic in the androgen producing adrenocortical H295R cell line downregulates DENND1A. V2 and CYP17A1 mRNA expression but has no effect on DENND1A. V1 mRNA expression. As mentioned in Materials and Methods, our attempts to transfect human theca cells with miR-130b-mimic were not successful because the transfection reagents were toxic to the cells. Consequently, to explore whether miR-130b-3p could mediate a reciprocal decrease in DENND1A. V2 mRNA expression we used a surrogate cell system; androgen-producing human H295R adrenocortical cells, which can be transfected with high efficiency. H295R cells express high levels of endogenous DENND1A. V2 mRNA under control non-stimulated conditions⁷¹. We have also previously reported that forced overexpression of DENND1A. V2 in H295R cells results in an increase in CYP17A1 gene expression and androgen biosynthesis⁷¹. The H295R cells were transfected with 75 μM miR-130b-3p mimic and non-specific Neg control mimic (Neg-mimic) as described in Materials and Methods. These experiments were repeated a minimum of 4 times. 48 h thereafter, mRNA was harvested and DENND1A. V2, DENND1A. V1, and CYP17A1 mRNA expression were quantitated by qRT-PCR. In FIG. 5A, cumulative results from adrenocortical H295R cells transfected with miR-130b-3p mimic demonstrates that DENND1A. V2 mRNA was significantly decreased in cultures treated under both control and forskolin-stimulated conditions, as compared to Neg-mimic transfected cells. In contrast, the miR-130b-3p mimic had no significant effect on DENND1A. V1 mRNA (FIG. 5B). CYP17A1 mRNA expression (FIG. 5C) was significantly suppressed in miR-130b-3p transfected cells compared to Neg mimic in forskolin treated cells. Forskolin treatment also increased CYP17A1 mRNA in Neg-mimic cells.

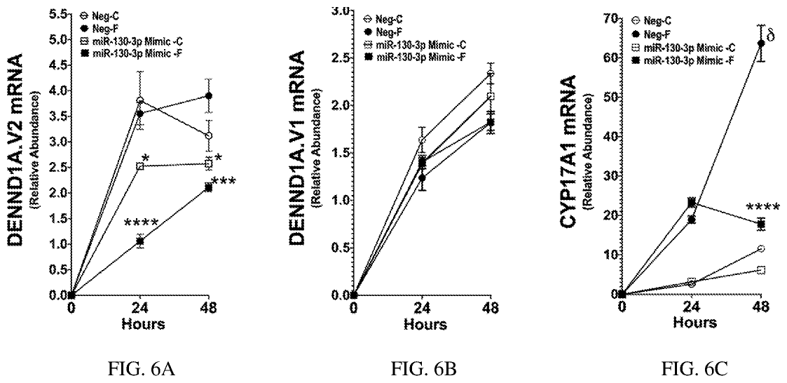

Differences in the time courses of DENND1A. V2 and CYP17A1 mRNA accumulation in adrenocortical H295R cells transfected with miR-130b-3p mimic. While our initial IPA and miR-TargetMiner analyses predicted that miR-130b-3p targets DENND1A (and specifically DENND1A. V2), we found no evidence to suggest that miR-130b-3p directly targets CYP17A1. To explore the possibility that miR-130 mimic has temporal effects on DENND1A. V2, DENND1A. V1, and CYP17A1 mRNA expression in H295 cells, H295R cells were transfected with 75 pM of miR-130b-3p mimic or non-specific Neg-mimic for 8 h, then subsequently treated in the absence (C) and presence of 20 μM forskolin (F) for 24 and 48 h. mRNA was harvested from the cells at both timepoints, and DENND1A. V2, DENND1A.1, and CYP17A1 mRNA accumulation was measured by qRT-PCR as described in Materials and Methods. In FIG. 6 we present cumulative data from 4 replicative experiments in H295R cells. In FIG. 6A, at both 24 and 48 h following treatment, miR-130b-3p mimic significantly suppresses DENND1A. V2 mRNA under both control non-stimulated and forskolin-stimulate conditions. In contrast, the time course of DENND1A. V1 accumulation (FIG. 6B) was unaffected by miR-130-3p mimic transfection. Of significant interest, CYP17A1 mRNA accumulation (FIG. 6C) was increased by forskolin at 24 and 48 h in Neg mimic transfected cells. Yet, while miR-130 mimic transfection had no significant effect on CYP17A1 mRNA following 24 hrs of treatment, at 48 h CYP17A1 mRNA was significantly suppressed (FIG. 6C). The finding that miR-130b-3p transfection, suppresses DENND1A. V2 mRNA at an earlier time point (24 h) than CYP17A1 mRNA (48 h); might suggest that miR-130b-3p mimic has direct effects on DENND1A. V2 mRNA, while CYP17A1 mRNA accumulation may be a result of indirect regulation via other intermediary factors and possibly via the effects of miR-130b-3p on DENND1A. V2.

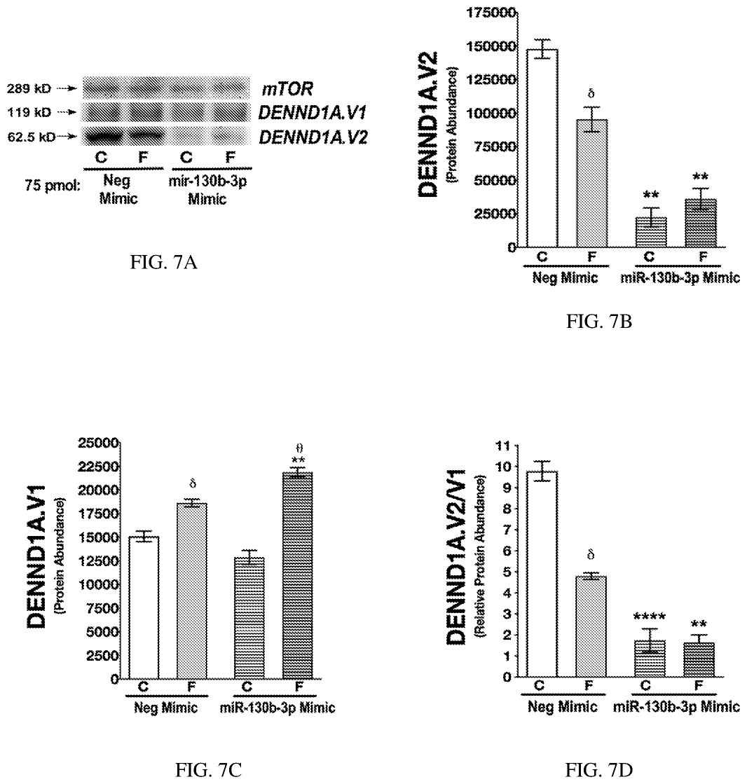

Western blot analysis of DENND1.V2 and DENND1A. V1 protein in H295R cells transfected with miR-130b-3p mimic. To explore the effects of overexpressing miR-130b-3p on DENND1A. V2 and DENND1A. V1 protein expression, Adrenocortical H295R cells were transiently transfected with 75 pM miR-130b-3p mimic and non-specific Neg mimic for 48 h in the presence (C) and absence of 20 μM forskloin (F). In FIG. 7A we present representative Western analysis of ˜62 kD DENND1A. V2 and ˜119 kD DENND1A. V1 in whole cell extracts isolated from H295R cells following 75 pM of Neg- and miR-130b-3p mimic transfections or non specific Neg-mimic. mTOR was used for total protein normalization. Quantitative Western Blot data from 4 replicate experiments of H295R cells transfected with 75 pM miR-130b-3p and Neg-mimics for 48 h, presented as the mean+/−SEM of the relative protein abundance normalized by mTOR of DENND1A. V2 (FIG. 7B) and DENND1A. V1 (FIG. 7C). DENND1A. V2 protein was significantly reduced following transfection with miR-130b-3p as compared to Neg-mimic under control and forskolin-stimulated conditions (FIG. 7B). In contrast, DENND1A. V1 protein was not significantly different following miR-mimic transfection in non-stimulated cells and was significantly increased by miR-130b-3p in forskolin treated cells (FIG. 7C). In FIG. 7D, the ratio of DENND1A. V2/V1 was observed to be significantly reduced following transfection with miR-130b-3p mimic under control and forskolin-stimulated conditions, as compared to Neg-mimic. Thus, over-expression of miR-130b-3p in H295R cells converts the cells from a PCOS phenotype of increased DENND1A. V2, to that of a normal cell phenotype of lower DENND1A. V2 expression. In FIG. 7D, both the relative abundance of DENND1A. V2 protein (FIG. 7B) and the ratio of DENND1A. V2/V1 protein decreased forskolin-stimulated H295R cells transfected with control Neg-mimic. Forskolin-treatment significantly increased DENND1A. V1 protein under both Neg- and miR-130b-3p mimic conditions (FIG. 7C). Forskolin treatment similarly augments DENND1A. V1 protein expression in normal and PCOS theca cells⁶¹.

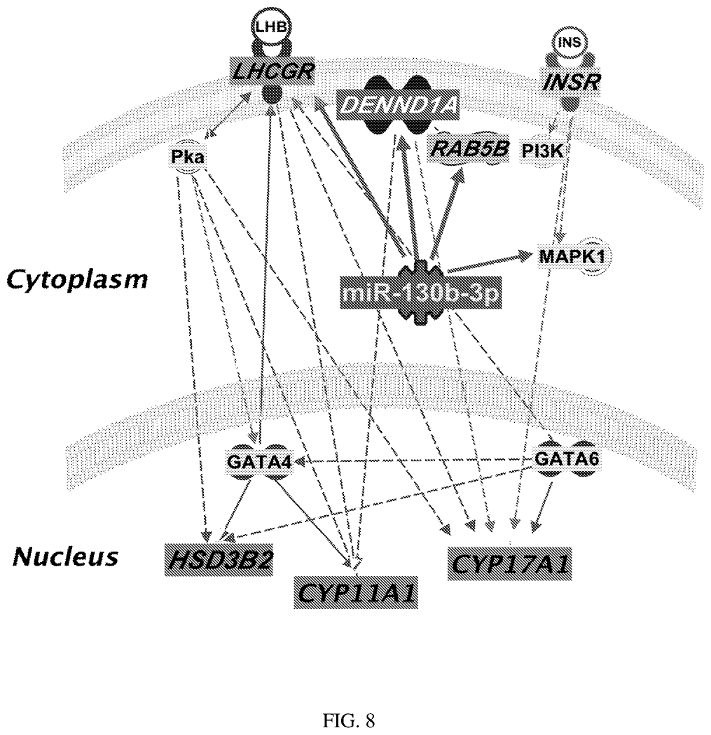

Ingenuity core pathway and network analyses suggests a common network between miR-130b-3p and PCOS GWAS candidate genes and signaling pathways expressed in normal and PCOS theca cells. The core pathway analyses tool in IPA identifies gene expression changes and the relationship to cellular processes. In contrast, IPA network analyses uses the data derived from core analyses to predict signaling pathways. To explore whether there is a common network between the three PCOS GWAS candidates that miR-130b-3p is known or highly-predicted to target, we utilized the “Grow” option which allowed us to identify any known or predicted connections in the current literature present in the Ingenuity Knowledge Base. We utilized the “Build” network function in IPA core analysis to create a cellular network. FIG. 8 depicts a diagram of the resulting IPA network between miR-130b-3p, DENND1A, two other PCOS GWAS candidates (i.e., LHCGR, RAB5B) as well as the signaling component MAPK1; known or highly predicted to be targets of miR-130b-3p. Also included are signaling components (PKA, AKT/PI3K) and transcription factors (GATA4/6) that have been previously reported to be involved in down-stream over-expression of steroidogenic enzyme gene expression (i.e., CYP17A1, cholesterol side-chain cleavage (CYP11A1), and 3β-HSD type II (HSD3B2)) which have been confirmed to be involved in excess androgen biosynthesis in PCOS theca cells. We have included the insulin receptor (INSR) in our diagram in FIG. 8, given that INSR mediated signaling has been widely accepted to be dysregulated in PCOS⁷⁴⁻⁷⁶, and the strong genetic association studies with the INSR^{75, 77.} The resulting theca cell network provides an innovative representation of the putative mechanisms by which decreased miR-130b-3p in PCOS theca cells may regulate the PCOS GWAS candidates DENND1A (DENND1A. V2), LHCGR, and RAB5B candidates and potentially mediate increased CYP17A1, CYP11A1, and HSD3B2 gene expression and hyperandrogenism in PCOS theca cells. As presented in Table 3, IPA core pathway and network analysis of miR-130b-3p's predicted interactions with the PCOS GWAS candidates (DENND1A. V2, LHCGR, RAB5B) as well as the signaling components MAPK1, PKA, AKT, and CYP17A1,3BHSD2, and CYP11A1, enabled us to identify significant functional and biological features with relevance to PCOS. For the associated network functions including endocrine system development and function, lipid metabolism, and small molecule biochemistry the IPA network score was 21 (Table 3A).

TABLE 3
Ingenuity Core Pathway Analysis of miR-130b-3p's highly predicted
targets which include DENND1A.V2, LHCGR, RAB5B, and MAPK1
signaling pathway. Key findings from core pathway analysis (performed
in IPA) of miR-130b-3p's predicted interactions with the 3 PCOS GWAS
candidates (DENND1A.V2, LHCGR, RAB5B), as well as the signaling
component MAPK1. Table 3 includes: Top Networks (3A), The Top
Canonical Pathways (3B), Top Diseases and Bio-functions (3C) and
Physiological System Development and Functions (3D).
3A Top Networks

| Name: | Score |
|---|---|
| Endocrine System Development and Function, Lipid | 21 |
| Metabolism, Small Molecule Biochemistry | |
| Organ Development, Cellular Growth and Proliferation, | 7 |
| Organ Morphology and Development | |

3B Top Canonical Pathways

| Name: | P-value | Overlap |
|---|---|---|
| Gs Signaling | 1.17E−05 | 2.8% |
| Ovarian Cancer Signaling | 3.59E−05 | 1.9% |
| cAMP-mediated signaling | 1.09E−44 | 1.3% |
| G-Protein Coupled Receptor Signaling | 1.94E−03 | 0.7% |
| Caveolar-mediated Endocytosis | 2.48E−03 | 0.6% |
| Signaling/Synaptogenesis Signaling | | |
| Pathway | | |
| Name: | P-value | Molecules |

3C Top Diseases and Bio-functions

| | | |
|---|---|---|
| Endocrine System Disorders | 1.29E−02-9.26E−06 | 7 |
| Organism Injury/Abnormalities | 4.97E−02-2.67E−06 | 9 |
| Reproductive System Disease | 4.87E−02-2.67E−06 | 7 |
| Cancer | 4.87E−02-2.67E−06 | 9 |
| Tumor Morphology | 4.48E−02-2.67E−46 | 3 |

3D Physiological System Development and Function

| | | |
|---|---|---|
| Embryonic Development | 8.04E−03-4.48E−04 | 3 |
| Organ Development | 2.48E−02-4.48E−04 | 3 |
| Organismal Development | 1.56E−02-4.48E−04 | 4 |
| Endocrine System Development and | 1.51E−02-3.21E−06 | 4 |
| Function | | |
| Organ Morphology | 2.00E−02-4.48E−04 | 3 |

### Discussion

PCOS is a multifactorial disorder that has been proposed to involve both genetic and epigenetic components, that can mediate changes in gene expression via transcriptional, post-transcriptional, translational, and post-translational mechanism(s). Recent GWAS in both Han Chinese and European populations identified candidate loci, some of which have been replicated by multiple laboratories, providing a basis for the molecular dissection of the pathophysiology of PCOS⁵²⁻⁵⁵. Theca cells, the androgen producing cells of the ovary and central to PCOS pathophysiology, also have evidence of an altered transcriptome profile in PCOS, which could be contributed by, at least in part, microRNA regulation^{29, 30}. Although there is one report of an examination of differential microRNA expression in normal and PCOS thecal tissues from wedge resections using a limited micro-array platform⁵¹, a detailed study of global miRNA expression using NGS high throughput small mRNA deep-sequencing combined with target gene analyses has not been performed in normal and PCOS theca cells. Altered microRNA levels have been associated with several phenotypes in the PCOS spectrum including diabetes, insulin resistance and inflammation. An increasing number of studies have shown differential miRNA expression in the ovarian stroma, follicular fluid, cumulus cells, granulosa cells, and endometrium of women with PCOS^{33-35, 43-45, 49, 78-80}. Our study is the first to examine differences in global microRNA expression in miRNA libraries that were sequenced and validated in detail, from well characterized theca cell cultures from PCOS and normal cycling women.

The first GWAS studies conducted on Han Chinese populations identified 11 loci with significant associations with PCOS^{52, 53}. Subsequent GWAS on European populations confirmed associations of some of the Han-Chinese GWAS to PCOS, and also identified additional candidate loci^{54, 55}. Together, four GWAS^{52, 53, 56-59} and PCOS GWAS meta-anlayses⁶⁰, have identified 22 candidate loci/genes for PCOS, including DENND1A. However, the functional relevance of the remaining loci with PCOS is not clear, and the exact molecular mechanism by which any of these candidate genes may contribute to the disorder is not known. Genotype-phenotype correlation studies of the susceptibility SNPs identified by GWAS have yielded low to modest relative risk ratios^{56, 81}. This is not unexpected for a complex disorder like PCOS and establishing a causal relationship between the genetic variants identified in GWAS and pathophysiological phenotype is challenging. However, it is important to recognize that while some PCOS GWAS gene loci consist of plausible PCOS candidate genes, no one has characterized the differential expression of the enormous number of mRNA transcripts and mRNA isoforms (>150) that are potentially encoded by the 22 GWAS loci in relevant normal and PCOS cells. Moreover, and the molecular mechanisms by which most of the identified genes contribute to PCOS are not clearly understood.

In the present study, miRNA expression profiles of human theca cell cultures established from women with PCOS and without the disease were determined using next generation small RNA deep-sequencing. The data derived from these studies demonstrated that over 1800 microRNAs are in normal and PCOS theca cells, and identified the 18 most statistically relevant differentially expressed miRNA in normal and PCOS theca cells (Table 1, FIG. 1). Of the statistically relevant 18 miRNA we observed to be differentially expressed in our normal and PCOS theca cells in culture (Table 1), only miR-502-3p was observed to overlap with those identified by the miRNA microarray platform utilized by Lin et al⁵¹, which may have not included the 18 miRNA we found to be significant. There was also no overlap between the 18 miRNA we identified and the differentially expressed miRNA reported in granulosa cells from normal cycling and PCOS women^{16, 39-41, 43, 82-86}, nor was there overlap with miRNA in human follicular fluid that were reported to be associated with PCOS³⁴. miR-23a and miR-23b were reported to be differentially expressed in plasma from normal cycling and PCOS women⁸⁷, however these miRNA were not significantly associated with PCOS based on our deep-sequencing study. While there are studies demonstrating that miR-93 is differentially expressed in many PCOS cells and plasma from normal cycling and PCOS women, with evidence for decreased expression in PCOS blastocysts and increased expression in PCOS adipocytes⁸⁸, granulosa cells⁸² as well as in the circulation⁸⁹, we did not find significant differences in miR-93 expression levels between normal PCOS theca cells in our deep sequencing or by Taqman-based qRT-PCR assays.

Subsequent target gene analysis of the differentially expressed miRNA was focused on PCOS candidate genes identified by GWAS⁵²⁻⁵⁵. In Table 2, target prediction analyses (following both IPA analyses, miR-Base, and mIR-Target Miner Data Base analyses) revealed that 4 of the 18 or 22% of these microRNAs (i.e., miR-130b-3p, miR-195-5p, and miR-654-5p, and miR-1271-5p), were shown to target 10 of the 22 (45%) of the PCOS GWAS candidate genes (i.e., DENND1A, INSR, LHCGR, RAB5B, ZNF217, YAP1, GATA4, HMGA2, ERBB3, and KCNA4), identified in Han-Chinese^{52, 53} and European populations^{54, 55}. miR-130b-3p was predicted to have target interactions with PCOS GWAS candidates DENND1A, ZNF217, RAB5B, LHCGR, ERBB3, and KCNA4 (Table 2).

We previously reported that a splice variant of DENND1A, one of the candidate genes identified in the Han Chinese GWAS and replicated in studies of women of European ancestry, is a functional GWAS candidate that mediates augmented androgen biosynthesis an associated gene expression in PCOS theca cells^{61, 71} More specifically, we found that the alternative truncated splice variant of DENND1A, termed DENND1A. V2 was augmented in PCOS theca cells and was found to mediate both increased CYP17A1 and CYP11A1 gene expression and excess androgen production in PCOS theca cells.

Here, we focused our studies on miR-130b-3p, given that it was predicted to target DENND1A, combined with our continued interest in examining the molecular basis of increased expression of the DENND1A isoform DENND1A. V2 in PCOS theca cells. Moreover, we were interested in exploring the post-translational mechanism(s) that underlie augmented hyperandrogenism in PCOS, and the possible role miR-130b-3p may play in DENND1A, as well as other relevant PCOS GWAS candidates.

The subsequent analyses of 3′UTR of DENND1A. V2 which identified a miR-130b-3p seed target sequence approximately 527 bp past the DENND1A. V2 CDS stop codon (ATTGA) (FIG. 3), provided further convincing evidence that miR-130b-3p plays a role in translational regulation and expression of DENND1A. V2. Our microRNA specific qRT-PCR analyses established that miR-130b-3p expression was down-regulated, while DENND1A. V2 expression was augmented in PCOS, as compared to normal theca cells (FIG. 2). Decreased miR-130b-3p was also found to be statistically correlated with increased DENND1A. V2 and CYP17A1 mRNA expression, as well as DHEA biosynthesis in PCOS theca cells, yet was not correlated with DENND1A. V1 mRNA expression (FIG. 4).

Our miR-130b-3p mimic studies confirmed the functional reciprocal relationship between miR-130b-3p and DENND1A. V2 and CYP17A1 mRNA expression (FIG. 5). Examination of the time course of miR-130b-3p transfection on DENND1A. V2, DENDD1A. V1 and CYP17A1 mRNA (FIG. 5) demonstrated that miR-130b-3p transfection suppresses DENND1A. V2 mRNA at an earlier time point (24 h) than CYP17A1 mRNA (48 h); and suggests that miR-130b-3p mimic has direct effects on DENND1A. V2 mRNA, while CYP17A1 mRNA accumulation may be a result of indirect regulation via other intermediary factors and possibly via the effects of miR-130b-3p on DENND1A. V2. The miR-130b-3p mimic studies also confirmed that the interaction of miR-130b-3p is specific for DENND1A. V2 and not DENND1A. V1 mRNA and protein expression (FIGS. 5-6). Decreased miR-130b-3p expression can possibly explain, at least in part, the DENND1A. V2 overexpression in PCOS theca cells as documented previously, which was not explained by any of the other genetic mechanisms^{61, 71}. These results suggest a translational mechanism for DENND1A. V2 overexpression in PCOS theca cells via miR-130b-3p regulation.

It is widely accepted that miRNA are usually involved in complex regulatory networks and their expression patterns are themselves regulated by several factors. As presented in FIG. 7, our ingenuity pathway analyses suggests there is a common network between miR-130b-3p, DENND1A, LHCGR, RAB5B, the signaling component MAPK1, and several signaling components (PKA, AKT/PI3K, cAMP) and transcription factors (GATA4/6) previously reported mediate changes in gene expression in PCOS theca cells, as well as being involved in down-stream over-expression of steroidogenic enzyme gene expression (i.e., CYP17A1, CYP11A1, and HSD3B2) which have been identified to mediate excess androgen biosynthesis in PCOS theca cells^{24, 61, 64, 65, 67}. The signaling components Pka, MAPK1, and PI3K have been widely reported to be associated with LHCGR and INSR dependent signaling. The transcription factors GATA4/6 included in our IPA Network have also been reported to be pertinent to steroidogenic enzyme gene expression and androgen biosynthesis⁴⁸. Collectively, these results also support our hypothesis that miR-130b-3p and DENND1A are involved in a complex network, in combination with PCOS GWAS candidates LHCGR, RAB5B, and can explain the ovarian hyperandrogenemia associated with and central to PCOS.

More in depth IPA core pathway and network analysis enabled us to identify significant functional and biological features with relevance to PCOS (Table 3). Several PCOS relevant features were identified in the top 5, A: Networks, B: Canonical Pathways, C: Diseases and Bio-functions, and D: Physiological System Development and Functions. Included in the top 5 Networks are endocrine system development and function, lipid metabolism, and small molecule biochemistry (Table 3A). Four of the top five Canonical Pathways are key signaling pathways central to ovarian function and PCOS pathophysiology including Gs signaling, cAMP signaling, G-protein coupled signaling, and ovarian cancer signaling (Table 3B). Although there is no firm evidence for associations between PCOS and ovarian cancers because of limited sample sizes and confounding risk factors, perturbation of pathways in ovarian cancer are important for normal ovarian function⁹⁰. The remaining top Canonical Pathways include: caveolar mediated endocytosis signaling, and the newly defined synaptogenesis signaling pathway. The top Diseases and Bio-functions includes an over-representation of diseases and disorders that are highly concordant with PCOS including endocrine system disorders, organismal injury and abnormalities, and reproductive system disease (Table 3C). The predicted relevance of the Physiology System Development and Function presented in Table 3D, with respect to embryonic, organ, organismal development, endocrine (reproductive) system development and function, and organ morphology, are highly reflective of the pathophysiological phenotypes associated with PCOS. These results suggest that more than one PCOS candidate gene is involved in pathophysiology of PCOS, which warrants detailed study of other GWAS candidate genes in a PCOS context⁹¹.

In this report we presented the first data to demonstrate that miR-130b-3p regulates the expression of a truncated isoform of the PCOS GWAS candidate DENND1A, DENND1A. V2 in normal and PCOS theca cells. The IPA theca cell cellular network we have presented, provides an innovative representation of the putative mechanism by which decreased miR-130b-3p in PCOS theca cells may regulate DENND1A. V2 and LHCGR, RAB5B, and the MAPK1, PI3K, cAMP/PKA signaling pathways and mediate increased CYP17A1, CYP11A1, and HSD3B2 gene expression and hyperandrogenism in PCOS theca cells.

### Example 2

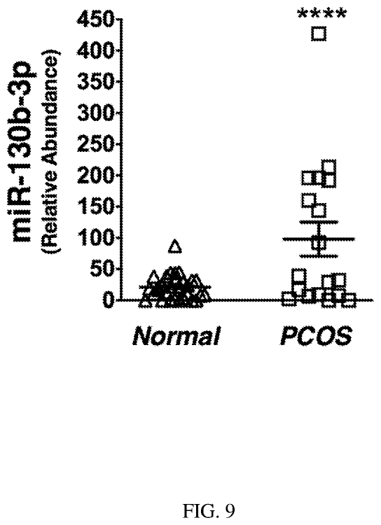

FIG. 9 demonstrates a differential abundance in serum of hsa-miR-130b-3p in PCOS versus normal women. Without being bound by theory, the directional differences in hsa-miR-130b-3p abundance between theca cells (down in PCOS) and serum (up in PCOS) suggests an intriguing regulatory mechanism(s) for intracellular homeostasis of this microRNA, including the export of the microRNA from organs/cells (i.e., adrenal, kidney, liver, ovarian granulosa or theca) to peripheral fluids in the context of PCOS.

### REFERENCES

1. Azziz R, Dumesic D A, Goodarzi M O. Polycystic ovary syndrome: an ancient disorder? Fertility and sterility. 2011; 95(5):1544-1548.
2. Knochenhauer E S, Key T J, Kahsar-Miller M, Waggoner W, Boots L R, Azziz R. Prevalence of the polycystic ovary syndrome in unselected black and white women of the southeastern United States: a prospective study. The Journal of clinical endocrinology and metabolism. 1998; 83(9):3078-3082.
3. Dunaif A. Polycystic ovary syndrome in 2011: Genes, aging and sleep apnea in polycystic ovary syndrome. Nature reviews Endocrinology. 2012; 8(2):72-74.
4. Legro R S, Arslanian S A, Ehrmann D A, et al. Diagnosis and treatment of polycystic ovary syndrome: an endocrine society clinical practice guideline. J Clin Endocrinol Metab. 2013; 98(12):4565-4592.
5. Azziz R. PCOS in 2015: New insights into the genetics of polycystic ovary syndrome. Nature reviews Endocrinology. 2016; 12(2):74-75.
6. Azziz R, Carmina E, Chen Z, et al. Polycystic ovary syndrome. Nat Rev Dis Primers. 2016; 2:16057.
7. Azziz R. Introduction: Determinants of polycystic ovary syndrome. Fertility and sterility. 2016; 106(1):4-5.
8. Diamanti-Kandarakis E, Dunaif A. Insulin resistance and the polycystic ovary syndrome revisited: an update on mechanisms and implications. Endocrine reviews. 2012; 33(6):981-1030.
9. Balen A, Homburg R, Franks S. Defining polycystic ovary syndrome. BMJ. 2009; 338:a2968.
10. Sung Y A, Oh J Y, Chung H, Lee H. Hyperandrogenemia is implicated in both the metabolic and reproductive morbidities of polycystic ovary syndrome. Fertility and sterility. 2014.
11. Moran L J, Misso M L, Wild R A, Norman R J. Impaired glucose tolerance, type 2 diabetes and metabolic syndrome in polycystic ovary syndrome: a systematic review and meta-analysis. Hum Reprod Update. 2010; 16(4):347-363.
12. Legro R, Driscoll D, Strauss III J, Fox A, Dunaif A. Evidence for a genetic basis for hyperandrogenemia in polycystic ovary syndrome. Proc Natl Acad Sci (USA). 1998; 95:14956-14960.
13. Legro R S, Strauss J F. Molecular progress in infertility: polycystic ovary syndrome. Fertility and sterility. 2002; 78(3):569-576.
14. Jiang L, Huang J, Chen Y, et al. Identification of several circulating microRNAs from a genome-wide circulating microRNA expression profile as potential biomarkers for impaired glucose metabolism in polycystic ovarian syndrome. Endocrine. 2016; 53(1):280-290.
15. Wu H L, Heneidi S, Chuang T Y, et al. The expression of the miR-25/93/106b family of micro-RNAs in the adipose tissue of women with polycystic ovary syndrome. The Journal of clinical endocrinology and metabolism. 2014; 99(12):E2754-2761.
16. Zhang C L, Wang H, Yan C Y, Gao X F, Ling X J. Deregulation of RUNX2 by miR-320a deficiency impairs steroidogenesis in cumulus granulosa cells from polycystic ovary syndrome (PCOS) patients. Biochem Biophys Res Commun. 2017; 482(4):1469-1476.
17. Ilie I R, Georgescu C E. Polycystic Ovary Syndrome-Epigenetic Mechanisms and Aberrant MicroRNA. Advances in clinical chemistry. 2015; 71:25-45.
18. Xu J, Bao X, Peng Z, et al. Comprehensive analysis of genome-wide DNA methylation across human polycystic ovary syndrome ovary granulosa cell. Oncotarget. 2016; 7(19):27899-27909.
19. Yu Y Y, Sun C X, Liu Y K, Li Y, Wang L, Zhang W. Genome-wide screen of ovary-specific DNA methylation in polycystic ovary syndrome. Fertility and sterility. 2015; 104(1):145-153 e146.0
20. Franks S, Webber L J, Goh M, et al. Ovarian morphology is a marker of heritable biochemical traits in sisters with polycystic ovaries. The Journal of clinical endocrinology and metabolism. 2008; 93(9):3396-3402.
21. Ehrmann D A, Rosenfield R L, Barnes R B, Brigell D F, Sheikh Z. Detection of functional ovarian hyperandrogenism in women with androgen excess. New England Journal of Medicine. 1992; 327(3):157-162.
22. Ehrmann D A, Barnes R B, Rosenfield R L. Polycystic ovary syndrome as a form of functional ovarian hyperandrogenism due to dysregulation of androgen secretion. Endocrine reviews. 1995; 16(3):322-353.
23. Nelson V L, Qin K N, Rosenfield R L, et al. The biochemical basis for increased testosterone production in theca cells propagated from patients with polycystic ovary syndrome. The Journal of clinical endocrinology and metabolism. 2001; 86(12):5925-5933.
24. Nelson V L, Legro R S, Strauss J F, 3rd, McAllister J M. Augmented androgen production is a stable steroidogenic phenotype of propagated theca cells from polycystic ovaries. Molecular endocrinology. 1999; 13(6):946-957.
25. Gilling-Smith C, Willis D S, Beard R W, Franks S. Hypersecretion of androstenedione by isolated thecal cells from polycystic ovaries. The Journal of clinical endocrinology and metabolism. 1994; 79(4):1158-1165.
26. Gilling-Smith C, Willis D S, Franks S. Oestradiol feedback stimulation of androgen biosynthesis by human theca cells. Human reproduction. 1997; 12(8):1621-1628.
27. Gilling-Smith C, Storey H, Rogers V, Franks S. Evidence for a primary abnormality in theca cell steroidogenesis in the polycystic ovarian syndrome. Clin Endocrinol. 1997; 47(93-99):1158-1165.
28. Franks S, White D, Gilling-Smith C, Carey A, Waterworth D, Williamson R. Hypersecretion of androgens by polycystic ovaries: the role of genetic factors in the regulation of cytochrome P450c17 alpha. Baillieres Clinical Endocrinology & Metabolism. 1996; 10(2):193-203.
29. Wood J R, Ho C K, Nelson-Degrave V L, McAllister J M, Strauss J F, 3rd. The molecular signature of polycystic ovary syndrome (PCOS) theca cells defined by gene expression profiling. J Reprod Immunol. 2004; 63(1):51-60.
30. Wood J R, Nelson V L, Ho C, et al. The molecular phenotype of polycystic ovary syndrome (PCOS) theca cells and new candidate PCOS genes defined by microarray analysis. The Journal of biological chemistry. 2003; 278(29):26380-26390.
31. Brower M A, Jones M R, Rotter J I, et al. Further investigation in europeans of susceptibility variants for polycystic ovary syndrome discovered in genome-wide association studies of Chinese individuals. The Journal of clinical endocrinology and metabolism. 2015; 100(1):E182-186.
32. Bartel D P. MicroRNAs: genomics, biogenesis, mechanism, and function. Cell. 2004; 116(2):281-297.
33. Roth L W, McCallie B, Alvero R, Schoolcraft W B, Minjarez D, Katz-Jaffe M G. Altered microRNA and gene expression in the follicular fluid of women with polycystic ovary syndrome. Journal of assisted reproduction and genetics. 2014; 31(3):355-362.
34. Sang Q, Yao Z, Wang H, et al. Identification of microRNAs in human follicular fluid: characterization of microRNAs that govern steroidogenesis in vitro and are associated with polycystic ovary syndrome in vivo. The Journal of clinical endocrinology and metabolism. 2013; 98(7):3068-3079.
35. Schmidt J, Weijdegard B, Mikkelsen A L, Lindenberg S, Nilsson L, Brannstrom M. Differential expression of inflammation-related genes in the ovarian stroma and granulosa cells of PCOS women. Molecular human reproduction. 2014; 20(1):49-58.
36. Wang M, Sun J, Xu B, et al. Functional Characterization of MicroRNA-27a-3p Expression in Human Polycystic Ovary Syndrome. Endocrinology. 2018; 159(1):297-309.
37. Moreno J M, Nunez M J, Quinonero A, et al. Follicular fluid and mural granulosa cells microRNA profiles vary in in vitro fertilization patients depending on their age and oocyte maturation stage. Fertility and sterility. 2015; 104(4):1037-1046 e1031.
38. Naji M, Aleyasin A, Nekoonam S, Arefian E, Mandian R, Amidi F. Differential Expression of miR-93 and miR-21 in Granulosa Cells and Follicular Fluid of Polycystic Ovary Syndrome Associating with Different Phenotypes. Sci Rep. 2017; 7(1):14671.
39. Naji M, Nekoonam S, Aleyasin A, et al. Expression of miR-15a, miR-145, and miR-182 in granulosa-lutein cells, follicular fluid, and serum of women with polycystic ovary syndrome (PCOS). Arch Gynecol Obstet. 2018; 297(1):221-231.
40. Cal G, Ma X, Chen B, et al. MicroRNA-145 Negatively Regulates Cell Proliferation Through Targeting IRS1 in Isolated Ovarian Granulosa Cells From Patients With Polycystic Ovary Syndrome. Reprod Sci. 2017; 24(6):902-910.
41. Fu X, He Y, Wang X, et al. MicroRNA-16 Promotes Ovarian Granulosa Cell Proliferation and Suppresses Apoptosis Through Targeting PDCD4 in Polycystic Ovarian Syndrome. Cell Physiol Biochem. 2018; 48(2):670-682.
42. Hou Y, Wang Y, Xu S, Qi G, Wu X. Bioinformatics identification of microRNAs involved in polycystic ovary syndrome based on microarray data. Mol Med Rep. 2019.
43. Chen B, Xu P, Wang J, Zhang C. The role of MiRNA in polycystic ovary syndrome (PCOS). Gene. 2019; 706:91-96.
44. Wang T, Liu Y, Lv M, et al. miR-323-3p regulates the steroidogenesis and cell apoptosis in polycystic ovary syndrome (PCOS) by targeting IGF-1. Gene. 2019; 683:87-100.
45. Zhai J, Yao G D, Wang J Y, et al. Metformin Regulates Key MicroRNAs to Improve Endometrial Receptivity Through Increasing Implantation Marker Gene Expression in Patients with PCOS Undergoing IVF/ICSI. Reprod Sci. 2019:1933719118820466.
46. Blandino G, Valerio M, Cioce M, et al. Metformin elicits anticancer effects through the sequential modulation of DICER and c-MYC. Nat Commun. 2012; 3:865.
47. Ye H, Li X, Zheng T, et al. The Hippo Signaling Pathway Regulates Ovarian Function via the Proliferation of Ovarian Germline Stem Cells. Cell Physiol Biochem. 2017; 41(3):1051-1062.
48. Fluck C E, Miller W L. GATA-4 and GATA-6 modulate tissue-specific transcription of the human gene for P450c17 by direct interaction with Sp1. Molecular endocrinology. 2004; 18(5):1144-1157.
49. Sorensen A E, Wissing M L, Salo S, Englund A L, Dalgaard L T. MicroRNAs Related to Polycystic Ovary Syndrome (PCOS). Genes (Basel). 2014; 5(3):684-708.
50. Ji S Y, Liu X M, Li B T, et al. The polycystic ovary syndrome-associated gene Yap1 is regulated by gonadotropins and sex steroid hormones in hyperandrogenism-induced oligo-ovulation in mouse. Molecular human reproduction. 2017; 23(10):698-707.
51. Lin L, Du T, Huang J, Huang L L, Yang D Z. Identification of differentially expressed microRNAs in the ovary of polycystic ovary syndrome with hyperandrogenism and insulin resistance. Chin Med J (Engl). 2015; 128(2):169-174.
52. Chen Z J, Zhao H, He L, et al. Genome-wide association study identifies susceptibility loci for polycystic ovary syndrome on chromosome 2p16.3, 2p21 and 9q33.3. Nat Genet. 2011; 43(1):55-59.
53. Shi Y, Zhao H, Shi Y, et al. Genome-wide association study identifies eight new risk loci for polycystic ovary syndrome. Nat Genet. 2012; 44(9):1020-1025.
54. Day F R, Hinds D A, Tung J Y, et al. Causal mechanisms and balancing selection inferred from genetic associations with polycystic ovary syndrome. Nat Commun. 2015; 6:8464.
55. Hayes M G, Urbanek M, Ehrmann D A, et al. Genome-wide association of polycystic ovary syndrome implicates alterations in gonadotropin secretion in European ancestry populations. Nat Commun. 2015; 6:7502.
56. Cui L, Zhao H, Zhang B, et al. Genotype-phenotype correlations of PCOS susceptibility SNPs identified by GWAS in a large cohort of Han Chinese women. Human reproduction. 2013; 28(2):538-544.
57. Hayes M G, Urbanek M, Ehrmann D A, et al. Corrigendum: Genome-wide association of polycystic ovary syndrome implicates alterations in gonadotropin secretion in European ancestry populations. Nat Commun. 2016; 7:10762.
58. Goodarzi M O, Jones M R, Li X, et al. Replication of association of DENND1A and THADA variants with polycystic ovary syndrome in European cohorts. J Med Genet. 2012; 49(2):90-95.
59. Pau C T, Mosbruger T, Saxena R, Welt C K. Phenotype and Tissue Expression as a Function of Genetic Risk in Polycystic Ovary Syndrome. PloS one. 2017; 12(1):e0168870.
60. Day F, Karaderi T, Jones M R, et al. Large-scale genome-wide meta-analysis of polycystic ovary syndrome suggests shared genetic architecture for different diagnosis criteria. PLoS Genet. 2018; 14(12):e1007813.
61. McAllister J M, Modi B, Miller B A, et al. Overexpression of a DENND1A isoform produces a polycystic ovary syndrome theca phenotype. Proceedings of the National Academy of Sciences of the United States of America. 2014; 111(15):E1519-1527.
62. McAllister J M. Functional, long-term human theca and granulosa cell cultures from polycystic ovaries. Endocrine. 1995; 3(2):143-149.
63. Nelson-Degrave V L, Wickenheisser J K, Hendricks K L, et al. Alterations in mitogen-activated protein kinase kinase and extracellular regulated kinase signaling in theca cells contribute to excessive androgen production in polycystic ovary syndrome. Molecular endocrinology. 2005; 19(2):379-390.
64. Wickenheisser J K, Biegler J M, Nelson-Degrave V L, Legro R S, Strauss J F, 3rd, McAllister J M. Cholesterol side-chain cleavage gene expression in theca cells: augmented transcriptional regulation and mRNA stability in polycystic ovary syndrome. PloS one. 2012; 7(11):e48963.
65. Wickenheisser J K, Quinn P G, Nelson V L, Legro R S, Strauss J F, 3rd, McAllister J M. Differential activity of the cytochrome P450 17alpha-hydroxylase and steroidogenic acute regulatory protein gene promoters in normal and polycystic ovary syndrome theca cells. The Journal of clinical endocrinology and metabolism. 2000; 85(6):2304-2311.
66. Strauss J F, 3rd. Some new thoughts on the pathophysiology and genetics of polycystic ovary syndrome. Ann N Y Acad Sci. 2003; 997:42-48.
67. Wickenheisser J K, Nelson-DeGrave V L, Quinn P G, McAllister J M. Increased cytochrome P450 17alpha-hydroxylase promoter function in theca cells isolated from patients with polycystic ovary syndrome involves nuclear factor-1. Molecular endocrinology. 2004; 18(3):588-605.
68. Nelson-DeGrave V L, Wickenheisser J K, Cockrell J E, et al. Valproate potentiates androgen biosynthesis in human ovarian theca cells. Endocrinology. 2004; 145(2):799-808.
69. Bani Mohammad M, Majdi Seghinsara A. Polycystic Ovary Syndrome (PCOS), Diagnostic Criteria, and AMH. Asian Pac J Cancer Prev. 2017; 18(1):17-21.
70. RRID:CVCL_0458). scicrunch.org/Rresolver/CVCL_0458). Accessed.
71. Tee M K, Speek M, Legeza B, et al. Alternative splicing of DENND1A, a PCOS candidate gene, generates variant 2. Molecular and cellular endocrinology. 2016; 434:25-35.
72. RRID:AB_2092106. scicrunch.org/resolver/AB_2092106. Accessed.
73. RRID:AB_330978. scicrunch.org/resolver/AB_330978. Accessed.
74. Li M, Youngren J F, Dunaif A, et al. Decreased insulin receptor (IR) autophosphorylation in fibroblasts from patients with PCOS: effects of serine kinase inhibitors and IR activators. The Journal of clinical endocrinology and metabolism. 2002; 87(9):4088-4093.
75. Urbanek M, Woodroffe A, Ewens K G, et al. Candidate gene region for polycystic ovary syndrome on chromosome 19p13.2. The Journal of clinical endocrinology and metabolism. 2005; 90(12):6623-6629.
76. Legro R S, Urbanek M, Kunselman A R, Leiby B E, Dunaif A. Self-selected women with polycystic ovary syndrome are reproductively and metabolically abnormal and undertreated. Fertility and sterility. 2002; 78(1):51-57.
77. Stewart D R, Dombroski B, Urbanek M, et al. Fine Mapping of Genetic Susceptibility to Polycystic Ovary Syndrome on Chromosome 19p13.2 and Tests for Regulatory Activity. The Journal of clinical endocrinology and metabolism. 2006.
78. Imbar T, Eisenberg I. Regulatory role of microRNAs in ovarian function. Fertility and sterility. 2014; 101(6):1524-1530.
79. Huang X, Liu C, Hao C, et al. Identification of altered microRNAs and mRNAs in the cumulus cells of PCOS patients: miRNA-509-3p promotes oestradiol secretion by targeting MAP3K8. Reproduction. 2016; 151(6):643-655.
80. Sorensen A E, Wissing M L, Englund A L, Dalgaard L T. MicroRNA Species in Follicular Fluid Associating With Polycystic Ovary Syndrome and Related Intermediary Phenotypes. The Journal of clinical endocrinology and metabolism. 2016; 101(4):1579-1589.
81. Zhao C, Liu X, Shi Z, et al. Role of serum miRNAs in the prediction of ovarian hyperstimulation syndrome in polycystic ovarian syndrome patients. Cell Physiol Biochem. 2015; 35(3):1086-1094.
82. Jiang L, Huang J, Li L, et al. MicroRNA-93 promotes ovarian granulosa cells proliferation through targeting CDKN1A in polycystic ovarian syndrome. The Journal of clinical endocrinology and metabolism. 2015; 100(5):E729-738.
83. Wang M, Liu M, Sun J, et al. MicroRNA-27a-3p affects estradiol and androgen imbalance by targeting Creb1 in the granulosa cells in mouse polycystic ovary syndrome model. Reprod Biol. 2017; 17(4):295-304.
84. Yao L, Li M, Hu J, Wang W, Gao M. MiRNA-335-5p negatively regulates granulosa cell proliferation via SGK3 in PCOS. Reproduction. 2018.
85. Yin M, Wang X, Yao G, et al. Transactivation of micrornA-320 by microRNA-383 regulates granulosa cell functions by targeting E2F1 and SF-1 proteins. The Journal of biological chemistry. 2014; 289(26):18239-18257.
86. Zhong Z, Li F, Li Y, et al. Inhibition of microRNA-19b promotes ovarian granulosa cell proliferation by targeting IGF-1 in polycystic ovary syndrome. Mol Med Rep. 2018; 17(4):4889-4898.
87. Xiong W, Lin Y, Xu L, et al. Circulatory microRNA 23a and microRNA 23b and polycystic ovary syndrome (PCOS): the effects of body mass index and sex hormones in an Eastern Han Chinese population. J Ovarian Res. 2017; 10(1):10.
88. McCallie B, Schoolcraft W B, Katz-Jaffe M G. Aberration of blastocyst microRNA expression is associated with human infertility. Fertility and sterility. 2010; 93(7):2374-2382.
89. Sathyapalan T, David R, Gooderham N J, Atkin S L. Increased expression of circulating miRNA-93 in women with polycystic ovary syndrome may represent a novel, non-invasive biomarker for diagnosis. Sci Rep. 2015; 5:16890.
90. Fauser B C, Tarlatzis B C, Rebar R W, et al. Consensus on women's health aspects of polycystic ovary syndrome (PCOS): the Amsterdam ESHRE/ASRM-Sponsored 3rd PCOS Consensus Workshop Group. Fertility and sterility. 2012; 97(1):28-38 e25.
91. Chen Y H, Heneidi S, Lee J M, et al. miRNA-93 inhibits GLUT4 and is overexpressed in adipose tissue of polycystic ovary syndrome patients and women with insulin resistance. Diabetes. 2013; 62(7):2278-2286.
92. Hyperandrogenism and Metabolic Syndrome Are Associated With Changes in Serum-Derived microRNAs in Women With Polycystic Ovary Syndrome. Sørensen A E, Udesen P B, Maciag G, Geiger J, Saliani N, Januszewski A S, Jiang G, Ma R C, Hardikar A A, Wissing M L M, Englund A L M, Dalgaard L T. Front Med (Lausanne). 2019 Nov. 1; 6:242. doi: 10.3389/fmed.2019.00242. eCollection 2019.
93. Non-targeted profiling of circulating microRNAs in women with polycystic ovary syndrome (PCOS): effects of obesity and sex hormones. Murri M, Insenser M, Fernández-Durán E, San-Millán J L, Luque-Ramírez M, Escobar-Morreale H F. Metabolism. 2018 September; 86:49-60. doi: 10.1016/j.metabol.2018.01.011. Epub 2018 Feb. 2.
94. The Role of Serum MicroRNA-6767-5p as a Biomarker for the Diagnosis of Polycystic Ovary Syndrome. Song D K, Sung Y A, Lee H. PLoS One. 2016 Sep. 27; 11(9):e0163756. doi: 10.1371/journal.pone.0163756. eCollection 2016.
95. MicroRNAs related to androgen metabolism and polycystic ovary syndrome. Sørensen A E, Udesen P B, Wissing M L, Englund A L M, Dalgaard L T. Chem Biol Interact. 2016 Nov. 25; 259(Pt A):8-16. doi: 10.1016/j.cbi.2016.06.008. Epub 2016 Jun. 5.
96. Identification of several circulating microRNAs from a genome-wide circulating microRNA expression profile as potential biomarkers for impaired glucose metabolism in polycystic ovarian syndrome. Jiang L, Huang J, Chen Y, Yang Y, Li R, Li Y, Chen X, Yang D. Endocrine. 2016 July; 53(1):280-90. doi: 10.1007/s12020-016-0878-9. Epub 2016 Feb. 9.)

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A method for diagnosis of a disorder that is positively correlated with expression of DENN/MADD domain containing 1A variant 2 (DENND1A. V2) mRNA and/or protein in a subject comprising:
measuring an expression level of at least one miRNA selected from the group consisting of miR-501-3p, miR-100-5p, miR-409-5p, miR-1271-5p, miR-1301-3p, miR-130b-3p, miR-99b-5p, miR-127-3p, miR-148b-5p, miR-654-5p, miR-195-5p, miR-744-5p, miR-1293, miR-410-3p, miR-4524a-5p, miR-502-3p, and miR-494-3p in a biological sample from said subject; and
comparing the expression level of the at least one miRNA to a corresponding reference value.

2. The method of claim 1, wherein the corresponding reference value is obtained from one or more healthy subjects, wherein the biological sample is not serum, and wherein the method further comprises
determining that the subject has the disorder when the expression level of one or more of miR-130b-3p, miR-148b-5p, miR-195-5p, and miR-4524a-5p is lower than the corresponding reference value; and/or
determining that the subject has the disorder when the expression level of one or more of miR-501-3p, miR-100-5p, miR-409-5p, miR-1271-5p, miR-1301-3p, miR-99b-5p, miR-127-3p, miR-654-5p, miR-744-5p, miR-1293, miR-410-3p, miR-502-3p, and miR-494-3p is higher than the corresponding reference value.

3. The method of claim 1, wherein the at least one miRNA includes miR-130b-3p.

4. The method of claim 3, wherein the corresponding reference value is obtained from one or more healthy subjects, the biological sample is serum, and wherein the method further comprises
determining that the subject has the disorder when the expression level of miR-130b-3p is higher than the corresponding reference value.

5. The method of claim 1, wherein the disorder is polycystic ovary syndrome (PCOS).

6. The method of claim 1, wherein the biological sample is blood, serum, urine, plasma, or saliva and/or a biopsy or surgical specimen of the ovary, endometrium, adipose tissue, or skeletal muscle.

7. A method for diagnosis of a disorder that is positively correlated with expression of DENND1A. V2 mRNA and/or protein in a subject comprising:
measuring an expression level of DENND1A. V2 mRNA and miR-130b-3p in a biological sample from said subject;
determining a ratio of the expression level of DENND1A. V2 mRNA to the expression level of miR-130b-3p; and
comparing the ratio to a corresponding reference ratio.

8. The method of claim 7, wherein the corresponding reference ratio is obtained from one or more healthy subjects, wherein the biological sample is not serum, and wherein the method further comprises
determining that the subject has the disorder when the ratio is higher than the corresponding reference ratio.

9. The method of claim 7, wherein the corresponding reference ratio is obtained from one or more healthy subjects, wherein the biological sample is serum, and wherein the method further comprises
determining that the subject has the disorder when the ratio is lower than the corresponding reference ratio.

10. The method of claim 7, wherein the disorder is PCOS.

11. The method of claim 7, wherein the biological sample is blood, serum, urine, plasma, or saliva and/or a biopsy or surgical specimen of the ovary, endometrium, adipose tissue, or skeletal muscle.

12. A method for the treatment of a disorder that is positively correlated with expression of DENND1A. V2 mRNA and/or protein comprising:
measuring an expression level of at least one miRNA selected from the group consisting of miR-501-3p, miR-100-5p, miR-409-5p, miR-1271-5p, miR-1301-3p, miR-130b-3p, miR-99b-5p, miR-127-3p, miR-148b-5p, miR-654-5p, miR-195-5p, miR-744-5p, miR-1293, miR-410-3p, miR-4524a-5p, miR-502-3p, and miR-494-3p in a biological sample from said subject;
comparing the expression level of the at least one miRNA to a corresponding reference value;
determining that the subject has the disorder when
(a) the biological sample is not serum and
the expression level of one or more of miR-130b-3p, miR-148b-5p, miR-195-5p, and miR-4524a-5p is lower than the corresponding reference value; and/or
the expression level of one or more of miR-501-3p, miR-100-5p, miR-409-5p, miR-1271-5p, miR-1301-3p, miR-99b-5p, miR-127-3p, miR-654-5p, miR-744-5p, miR-1293, miR-410-3p, miR-502-3p, and miR-494-3p is higher than the corresponding reference value; or
(b) the biological sample is serum and
the expression level of one or more of miR-130b-3p, miR-148b-5p, miR-195-5p, and miR-4524a-5p is higher than the corresponding reference value; and/or
the expression level of one or more of miR-501-3p, miR-100-5p, miR-409-5p, miR-1271-5p, miR-1301-3p, miR-99b-5p, miR-127-3p, miR-654-5p, miR-744-5p, miR-1293, miR-410-3p, miR-502-3p, and miR-494-3p is lower than the corresponding reference value; and
administering to the subject determined to have the disorder an appropriate treatment for the disorder.

13. The method of claim 12, wherein the at least one miRNA includes miR-130b-3p.

14. The method of claim 12, wherein the disorder is PCOS.

15. The method of claim 14, wherein the treatment is selected from the group consisting of hormonal therapy, an insulin-sensitizing agent, an agent or surgical intervention to increase fertility, an antibody or an antigen binding fragment thereof that specifically recognizes DENND1A. V2 protein, and a RNAi containing composition, wherein said RNAi composition decreases the expression of DENND1A. V2 or other PCOS gene products.

16. The method of claim 15, wherein the RNAi containing composition comprises miRNA or a miRNA mimic.

17. The method of claim 12, wherein the biological sample is blood, serum, urine, plasma, or saliva and/or a biopsy or surgical specimen of the ovary, endometrium, adipose tissue, or skeletal muscle.

18. A method for the treatment of a disorder that is positively correlated with expression of DENND1A. V2 mRNA and/or protein comprising:
measuring an expression level of DENND1A. V2 mRNA and miR-130b-3p in a biological sample from said subject;
determining a ratio of the expression level of DENND1A. V2 mRNA to the expression level of miR-130b-3p;
comparing the ratio to a corresponding reference ratio;
determining that the subject has the disorder when
(a) the biological sample is not serum and the ratio is higher than the corresponding reference ratio; or
(b) the biological sample is serum and the ratio is lower than the corresponding reference ration; and
administering to the subject determined to have the disorder an appropriate treatment for the disorder.

19. The method of claim 18, wherein the disorder is PCOS.

20. The method of claim 19, wherein the treatment is selected from the group consisting of hormonal therapy, an insulin-sensitizing agent, an agent or surgical intervention to increase fertility, an antibody or an antigen binding fragment thereof that specifically recognizes DENND1A. V2 protein, and a RNAi containing composition, wherein said RNAi composition decreases the expression of DENND1A. V2 or other PCOS gene products.

21. The method of claim 20, wherein the RNAi containing composition comprises miRNA or a miRNA mimic.

22. The method of claim 18, wherein the biological sample is blood, serum, urine, plasma, or saliva and/or a biopsy or surgical specimen of the ovary, endometrium, adipose tissue, or skeletal muscle.
